(19) United States
(12) Patent Application Publication
(10) Pub. No.: US 2025/0387187 A1
(43) Pub. Date: Dec. 25, 2025

(54) METHODS FOR ABDOMINAL SURGERY

(71) Applicant: Momentis Surgical Ltd., Or-Yehuda (IL)

(72) Inventors: Dvir COHEN, Or-Yehuda (IL); Michael CONDITT, Or-Yehuda (IL); lnbal REGEV, Or-Yehuda (IL)

(21) Appl. No.: 18/879,782

(22) PCT Filed: Jun. 30, 2023
(86) PCT No.: PCT/IB2023/056825

§ 371 Date: Dec. 29, 2024

Related U.S. Application Data
(60) Provisional application No. 63/357,274, filed on Jun. 30, 2022.

(51) Int. Cl. A61B 34/00; A61B 17/00; A61B 17/34; A61B 34/30

(52) U.S. Cl. CPC A61B 34/70; A61B 17/00234; A61B 17/3403; A61B 34/30; A61B 2017/00238; A61B 2017/00292; A61B 2017/3405; A61B 2034/302

(57) ABSTRACT

A method of conducting abdominal surgery in a surgical zone. The method includes creating an incision at a sub-Pfannenstiel region of the abdomen. A mechanical limb is inserted into a body of a subject through the incision, where the mechanical limb comprises a linear portion, at least part of which extends outside the body, and a tool coupled to a distal end of the mechanical limb. The mechanical limb is manipulated within the body so that the tool contacts the abdominal target site. The manipulating of the mechanical limb does not affect any portion of the mechanical limb disposed outside the body of the subject. Using the tool, the abdominal surgery is conducted within the surgical zone.

### REFERENCE TO RELATED APPLICATIONS

The present application gains priority from U.S. Provisional Patent Application No. 63/357,274 filed Jun. 30, 2022, entitled METHODS FOR ABDOMINAL SURGERY, which is incorporated by reference as if fully set forth herein.

### FIELD OF THE INVENTION

The present invention, in some embodiments thereof, relates to methods of key-hole abdominal surgery, and more particularly to methods of key-hole abdominal surgery using articulated mechanical limbs, which is performed through a Pfannenstiel incision, a sub-Pfannenstiel incision, or an umbilical incision.

### BACKGROUND OF THE INVENTION

For many years, surgeons have been seeking ways to reduce the level of invasiveness, scarring, and recovery times associated with surgical procedures. Over the last few decades, various robotic and laparoscopic surgical systems have been developed, which allow surgeons to conduct surgery, using robotic arms, catheters, and the like. Use of such robotic and laparoscopic surgical systems has reduced the size of the incisions required for conducting surgery, and thus has decreased scarring and recovery times. Some examples of robotic surgery systems are described, for example, in U.S. Pat. No. 9,788,911 and U.S. Patent Application Publication No. 2016/0184033.

With respect to abdominal surgery, it is known in the medical arts that incisions in the abdomen may lead to the formation of a hernia, or become infected. As such, it is desireable to reduce the number and/or dimension of incisions in the abdomen. Further, there are specific locations in the abdomen which are more suitable for making incisions, since they are less susceptible to infection and/or to the occurrence of hernias. These include an incision along the Pfannenstiel line, also known as the bikini line, or an incision within the umbilicus.

Some prior art robotic systems use stick-arms terminating in a suitable surgical tool. These arms have minimal flexibility, and must be inserted into the body at a specific angle, suitable for conducting of the surgery. If multiple stick-arms are used, multiple incisions are required in order to enable each of the stick-arms to work, in the desired location and angle. As such, while the incisions used for such robotic surgery are small, the use of multiple such incisions may be problematic because of the increased risk of infection and/or occurrence of hernias.

One may have attempted to conduct robotic surgery, using stick-arms as known in the art, via a Pfannenstiel or umbilical incision, in order to reduce the risk of infection. However, there are certain types of surgeries that cannot possibly be conducted in this manner, because of the flexibility limitations of the stick-arms.

There is thus a need in the art for a robotic or laparoscopic system capable of conducting abdominal surgical processes, throughout the entire abdominal cavity, while reducing the risk of hernias or infection by using a single, small, Pfannenstiel, sub-Pfannenstiel, or umbilical incision for insertion of the mechanical limbs used to conduct the surgical procedure.

### SUMMARY OF THE INVENTION

The present invention, in some embodiments thereof, relates to methods of key-hole abdominal surgery, and more particularly to methods of key-hole abdominal surgery using articulated mechanical limbs, which is performed via a Pfannenstiel incision, a sub-Pfannenstiel incision, or an umbilical incision.

In the following description and claims, any instance which refers to conducting surgery via a Pfannenstiel is considered to relate also to conducting the surgery via a sub-Pfannenstiel incision.

A “sub-Pfannenstiel incision”, in the context of the following description and claims, relates to an incision located below the Pfannenstiel line, anywhere between the Pfannenstiel line and the pubic bone.

An incision is considered to be “in close proximity” to another body part if a shortest distance, from the incision to the body part, measured along an external contour of the body, is at most one finger's breadth.

In accordance with some embodiments, there is provided a method of repairing a hernia, the method including:
inserting an articulated mechanical limb into a body through an abdominal incision, where the articulated mechanical limb includes:
   a linear portion, at least part of which extends outside the body, the linear portion being coupled to a mount disposed outside the body;
   at least one flexible portion coupled to the linear portion; and
   a tool coupled to the at least one flexible portion;
bending the articulated mechanical limb within the body at the at least one flexible portion so that the tool contacts the hernia, wherein that a sum of angles between adjacent effective segment longitudinal axes, in at least one three dimensionally oriented plane, is greater than 90°; and
repairing the hernia with the tool of the articulated mechanical limb, where the repairing includes covering the site of the hernia with a mesh.

In accordance with some embodiments, there is a provided a method of carrying out abdominal surgery in a surgical zone of a body, the method including:
inserting two mechanical limbs into a body through a single abdominal incision, each of the mechanical limbs terminating in a tool, where at least one of the two mechanical limbs is an articulated mechanical limb, including:
   a linear portion, at least part of which extends outside the body, the linear portion being coupled to a mount disposed outside the body; and
   at least one flexible portion coupled to the linear portion,
   wherein the tool is coupled to the second flexible portion;
bringing the tools of the mechanical limbs to the surgical zone, wherein the bringing includes bending the at least one articulated mechanical limb within the body at the at least one flexible portion such that the tool thereof enter the surgical zone, wherein a sum of angles between adjacent effective segment longitudinal axes, in at least one three dimensionally oriented plane, is greater than 90°; and
using the tools of the two mechanical limbs, conducting surgery within the surgical zone.

In accordance with some embodiments, there is provided a method of carrying out abdominal surgery, the method including:
inserting an articulated mechanical limb into a body through a Pfannenstiel incision or through a sub-Pfannenstiel incision, where the articulated mechanical limb includes:
   a linear portion, at least part of which extends outside the body, the linear portion being coupled to a mount disposed outside the body;
   at least one flexible portion coupled to the linear portion; and
   a tool coupled to the at least one flexible portion;
bending the mechanical limb within the body at the at least one flexible portion so that the tool thereof enters an abdominal surgical zone, the abdominal surgical zone being in a lower quadrant of the abdomen; and
using the tool, conducting surgery within the abdominal surgical zone.

In accordance with some embodiments, there is provided a method of carrying out abdominal surgery in a surgical zone of a body, the method including:
inserting an articulated mechanical limb into the body through an abdominal incision, the abdominal incision being outside of the surgical zone, where the articulated mechanical limb includes:
   a linear portion, at least part of which extends outside the body, the linear portion being coupled to a mount disposed outside the body;
   at least one flexible portion coupled to the linear portion; and
   a tool coupled to the at least one flexible portion;
bending the articulated mechanical limb within the body at the at least one flexible portion such that the tool thereof enters the surgical zone; and
using the tool, conducting surgery within the surgical zone,
wherein a shortest distance between the surgical zone and the abdominal incision, measured along a body wall, is less than 10 cm, less than 8 cm, less than 5 cm, less than 4 cm, less than 3 cm, less than 2 cm, or less than 1 cm.

In accordance with some embodiments, there is provided a method of carrying out abdominal surgery in a surgical zone of a body, the method including:
inserting an articulated mechanical limb into the body through an abdominal incision, the abdominal incision being outside of the surgical zone, where the articulated mechanical limb includes:
   a linear portion, at least part of which extends outside the body, the linear portion being coupled to a mount disposed outside the body;
   at least one flexible portion coupled to the linear portion; and
   a tool coupled to the at least one flexible portion;
bending the articulated mechanical limb within the body at the at least one flexible portion such that the tool thereof enters the surgical zone; and
using the tool, conducting surgery within the surgical zone,
wherein a ratio between a length of the articulated mechanical limb within the body, measured as a sum of lengths of longitudinal axes of portions of the articulated mechanical limb within the body, and a distance between the abdominal incision and the surgical zone, is at least 2:1, the distance being a shortest distance between the abdominal incision and the surgical zone when measured along a body wall.

In accordance with some embodiments, there is provided a method of carrying out abdominal surgery in a surgical zone of a body, the method including:
inserting an articulated mechanical limb into the body through an abdominal incision, where the articulated mechanical limb includes:
   a linear portion, at least part of which extends outside the body, the linear portion being coupled to a mount disposed outside the body;
   at least one flexible portion coupled to the linear portion; and
   a tool coupled to the at least one flexible portion;
bending the articulated mechanical limb within the body at the at least one flexible portion such that the tool thereof enters the surgical zone, wherein, following the bending, the tool is disposed above the horizon relative to the linear portion entering the abdominal incision; and
using the tool, conducting surgery within the surgical zone.

In accordance with some embodiments, there is provided a method of carrying out abdominal surgery in a surgical zone of a body, the method including:
inserting an articulated mechanical limb into the body through an abdominal incision, where the articulated mechanical limb includes:
   a linear portion, at least part of which extends outside the body, the linear portion being coupled to a mount disposed outside the body;
   at least one flexible portion coupled to the linear portion; and
   a tool coupled to the at least one flexible portion;
bending the articulated mechanical limb within the body at the at least one flexible portion such that the tool thereof enters the surgical zone, wherein, following the bending, the tool is disposed in a tool plane, the tool plane being above an incision plane formed by the abdominal incision and the linear portion, relative to a reference surface; and
using the tool, conducting surgery within the surgical zone.

In accordance with some embodiments, there is provided a method of carrying out abdominal surgery in a surgical zone of a body, the method including:
inserting an articulated mechanical limb into the body through an abdominal incision, where the articulated mechanical limb includes:
   a linear portion, at least part of which extends outside the body, the linear portion being coupled to a mount disposed outside the body;
   at least one flexible portion coupled to the linear portion; and
   a tool coupled to the at least one flexible portion;
bending the articulated mechanical limb within the body at the at least one flexible portion such that the tool thereof enters the surgical zone; and
using the tool, conducting surgery within the surgical zone,
wherein the inserting includes inserting the first and second flexible portions into the body, while maintaining at least a majority of the linear portion outside of the body.

In some embodiments, the inserting includes inserting the first and second flexible portions into the body, while maintaining the entirety of the linear portion outside of the body.

In accordance with some embodiments, there is provided a method of carrying out abdominal surgery in a surgical zone of a body, the method including:
inserting an articulated mechanical limb into the body through an abdominal incision, where the articulated mechanical limb includes:
   a linear portion, at least part of which extends outside the body, the linear portion being coupled to a mount disposed outside the body;
   at least one flexible portion coupled to the linear portion; and
   a tool coupled to the at least one flexible portion;
bending the articulated mechanical limb within the body at the at least one flexible portion such that the tool thereof enters the surgical zone; and
using the tool, conducting surgery within the surgical zone,
wherein a ratio between a length of the articulated mechanical limb disposed outside the body and a length of the articulated mechanical limb disposed within the body, measured as a sum of lengths of longitudinal axes of portions of the articulated mechanical limb within the body, is in the range of 1:2-1:4.

In accordance with some embodiments, there is provided a method of carrying out abdominal surgery in a surgical zone of a body, the method including:
inserting an articulated mechanical limb into the body through an abdominal incision, where the articulated mechanical limb includes:
   a linear portion, at least part of which extends outside the body, the linear portion being coupled to a mount disposed outside the body;
   at least one flexible portion coupled to the linear portion; and
   a tool coupled to the at least one flexible portion;
bending the articulated mechanical limb within the body at the at least one flexible portion such that the tool thereof enters the surgical zone; and
using the tool, conducting surgery within the surgical zone, wherein a ratio between a length of portions of the articulated mechanical limb disposed outside the body and a length of portions of the articulated mechanical limb disposed within the body, measured as a sum of lengths of longitudinal axes of portions of the articulated mechanical limb within the body, is at least 1:2 or at least 1:3.

In accordance with some embodiments, there is provided a method of carrying out abdominal surgery in a surgical zone of a body, the method including:
inserting an articulated mechanical limb into the body through an abdominal incision, where the articulated mechanical limb includes:
   a linear portion, at least part of which extends outside the body, the linear portion being coupled to a mount disposed outside the body;
   at least one flexible portion coupled to the linear portion; and
   a tool coupled to the at least one flexible portion;
bending the articulated mechanical limb within the body at the at least one flexible portion such that the tool thereof enters the surgical zone; and
using the tool, conducting surgery within the surgical zone, wherein a ratio between a length of the articulated mechanical limb, disposed within the body, which is stationary relative to the abdominal incision, and a length of the articulated mechanical limb disposed within the body which is movable relative to the abdominal incision, when the mount outside the body is stationary, is at least 1:5, at least 1:8, at least 1:10, or at least 1:15.

In accordance with some embodiments, there is provided a method of carrying out abdominal surgery using a mechanical limb including (i) a linear portion, (ii) at least one flexible portion coupled to the linear portion, and (iii) a tool coupled to the at least one flexible portion, the method including:
placing a trocar in the abdominal incision, while the trocar is in a first angular orientation relative to abdominal tissue including the incision;
inserting a mechanical limb into a body through the trocar to a first extent while the trocar is in the first angular orientation, such that a first segment of the mechanical limb is disposed within the body;
changing an angular orientation of the trocar, together with the first segment of the mechanical limb, relative to the abdominal tissue;
inserting the mechanical limb through the trocar to a second extent while the trocar is in the second angular orientation, such that the first segment and an additional second segment of the mechanical limb are disposed within the body;
bending the mechanical limb within the body at the first and the second flexible portions such that the tool thereof enters an abdominal surgical zone; and
using the tool, conducting surgery within the abdominal surgical zone.

In accordance with some embodiments, there is provided a method of conducting abdominal surgery in a surgical zone, the method including:
creating an incision at a sub-Pfannenstiel region of the abdomen;
inserting a mechanical limb into a body of a subject through the incision, where the mechanical limb includes a linear portion, at least part of which extends outside the body, and a tool coupled to a distal end of the mechanical limb;
manipulating the mechanical limb within the body so that the tool contacts the abdominal target site, where the manipulating of the mechanical limb does not affect any portion of the mechanical limb disposed outside the body of the subject; and
using the tool, conducting the abdominal surgery within the surgical zone.

In some embodiments, the creating of the incision includes creating the incision in close proximity to the pubic bone, below the Pfannenstiel line.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE FIGURES

The foregoing discussion will be understood more readily from the following detailed description of the invention, when taken in conjunction with the accompanying FIGS. 1A-11, in which:

FIGS. 1A, 1B, and 1C are simplified schematic views of articulated mechanical arms of a surgical device, in different orientations, according to some embodiments of the invention;

FIG. 2 is a simplified schematic view of a system where a device is held by a support in a position suitable for trans-Pfannenstiel surgery, according to some embodiments of the invention;

FIGS. 3A and 3B are simplified schematic views of a system where a device is held by a support in a position suitable for trans-umbilical surgery, according to some embodiments of the invention;

FIGS. 4A and 4B are schematic views of a position of a device of a system while conducting surgery for treatment of an inguinal hernia, via a Pfannenstiel incision, according to some embodiments;

FIGS. 5A and 5B are schematic views of a position of a device of a system while conducting surgery for treatment of a ventral hernia, via a Pfannenstiel incision, according to some embodiments;

FIG. 6 is a schematic view of a device of a system while conducting surgery, according to some embodiments;

FIG. 7 is a schematic view of a device of a system while conducting surgery, according to some embodiments;

FIGS. 8A, 8B, and 8C are schematic views of steps of insertion of a device of a system into the abdomen while conducting surgery, according to some embodiments;

FIGS. 9A, 9B, 9C, 9D, and 9E are schematic views of a system during conducting abdominal surgery via an abdominal incision, according to some embodiments;

FIGS. 10A, 10B, 10C, 10D, and 10E are schematic views of a method for conducting abdominal surgery via an abdominal incision, according to some embodiments; and

FIG. 11 is a flow chart of a method of conducting surgery for treatment of a hernia, according to some embodiments.

### DESCRIPTION OF THE PREFERRED EMBODIMENTS

The principles of the inventive methods of robotic and/or laparoscopic abdominal surgery, may be better understood with reference to the drawings and the accompanying description.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

For the purposes of this application, the terms “subject” and “patient” are used interchangeably, and relate to a human.

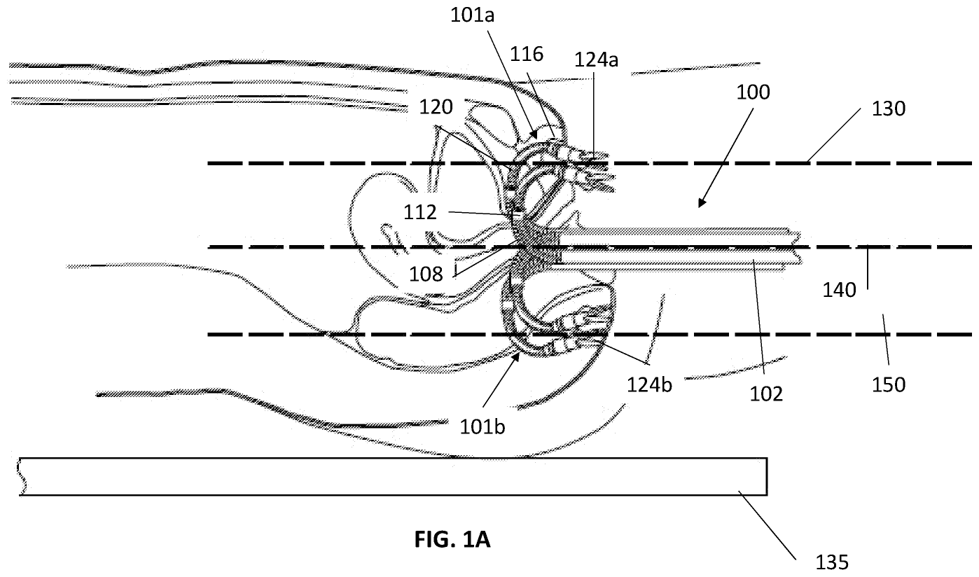

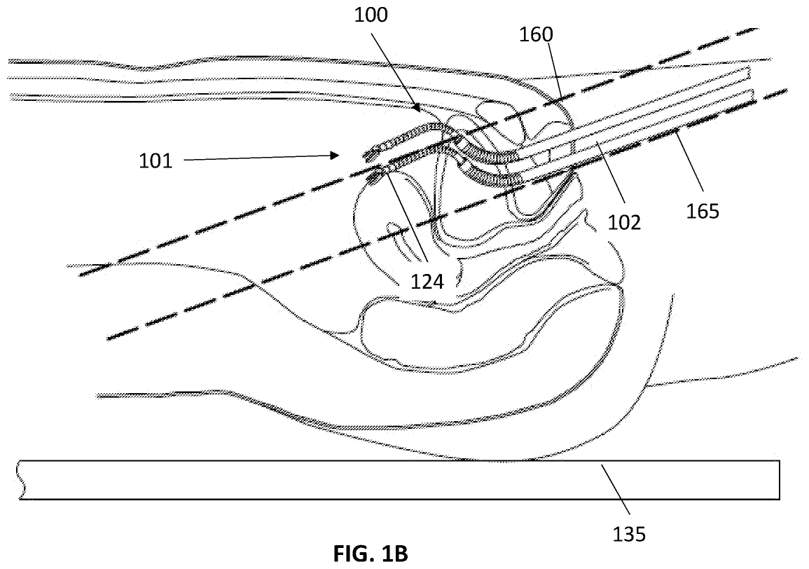

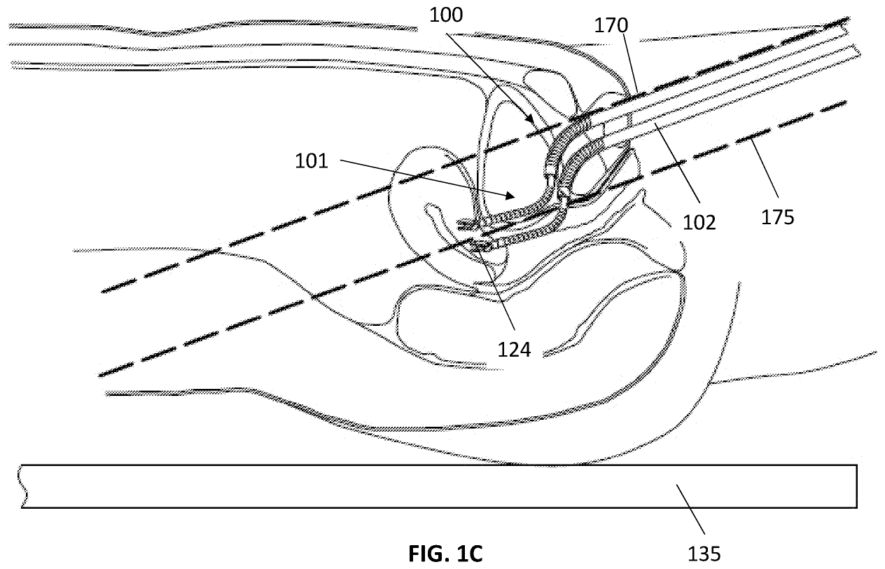

For the purposes of this application, the term “retroflex” relates to bending of a mechanical limb to an angle greater than 90 degrees, such that a vector at which the tip of the mechanical limb faces, has at least a component facing toward a base of the mechanical limb. Examples of retroflex bending of a mechanical limb are provided in FIG. 1A.

For the purposes of this application, the term “antigrade” relates to bending of a mechanical limb such that a vector representing the direction at which the tip of the mechanical limb faces, has at least a component facing away from a base of the mechanical limb. Examples of antigrade bending of a mechanical limb are provided in FIGS. 1B and 1C.

FIGS. 1A, 1B, and 1C are simplified schematic views of articulated mechanical arms of a surgical device, in different orientations, according to some embodiments of the invention.

As seen, a device 100 of FIGS. 1A to 1C (e.g. surgical device) includes at least one articulated mechanical limb 101, or arm, according to some embodiments of the invention. In some embodiments, the device includes a first arm and a second arm. However, the device may include more than two arms. In the context of the present application, an articulated mechanical arm, or limb, is a flexible arm made of two or more links, which can be manipulated to change the shape, form, or geometry of the arm.

In some embodiments each arm 101 includes a support segment 102, coupled to a first segment 112 by a first connecting section 108. First segment 112 may be coupled to a second segment 116 by a second connecting section 120=, and a third segment 124 may be coupled to second segment 116 by a third connecting section 128.

In some embodiments, support segment 102 is rigid. In some embodiments, support segment 102 may be flexible or include a flexible portion. In some embodiments, support segment 102 is linear.

In some embodiments, the articulated mechanical limb may include a humanoid like structure. For clarity, within this application, some device segments and connecting sections may be referred to by anatomical names:
support segment 102 is also termed torso 102;
first connecting section 108 is termed shoulder joint 108;
first segments 112 is also termed humerus 112;
second connecting section 120 is also termed elbow joint 120;
second segment 116 is also termed radius 116; and
third segment 124 is also termed hand tool 124.

In some embodiments, one or more connecting sections include a hinge. In some embodiments, one or more connecting sections are flexible and/or include a flexible portion. In an exemplary embodiment, for example, a mechanical articulated arm of the device includes an elbow joint and a shoulder joint. Bending of each joint may be distributed along the joint in a direction of a joint long axis.

In some embodiments, one or more device segments have a substantially cylindrical external shape (e.g. radius, humerus). In some embodiments, joints may have a circular cross-section perpendicular to a longitudinal axis of the joint. Alternatively, in some embodiments, one or more device segments and/or joints have a non-circular cross section or external shape, for example, oval, square, rectangular, or having an irregular shape.

In some embodiments, an articulated mechanical arm includes one or more short and/or adjustable segments. In some embodiments, flexible portions are directly connected.

In some embodiments, a mechanical articulated arm 101 of device 100 has at least the freedom of movement of human arms. Generally, segments of human limbs (e.g. arms, legs) move by flexion and extension from a proximal segment joint, and rotation around the proximal segment joint. For example, a human radius flexes and extends at the elbow and rotates around the elbow.

The term proximal joint herein refers to the joint which is least removed from the torso to which a segment is coupled, e.g. a hand proximal joint is the wrist, a radius proximal joint is the elbow joint, a humerus proximal joint is the shoulder joint.

The term proximal segment herein refers to the segment which is least removed from the torso to which a segment is coupled (e.g. by a proximal segment joint). For example, a hand proximal segment is the radius, a radius proximal segment is the humerus, a humerus proximal segment is the torso.

In some embodiments, one or more joints are unidirectionally bendable and extendable. In some embodiments, segment rotation around a segment proximal joint is achieved by rotation of a proximal segment around a proximal segment longitudinal axis. For example, rotation of the hand around the wrist joint is by rotation of the radius around a radius longitudinal axis.

Generally, human freedom of movement for arms includes limits to the angles of rotation and flexion. Optionally, in some embodiments, the device is restricted to human freedom of movements e.g. during one or more control modes.

In some embodiments, humerus 112 is adapted to flex and extend at shoulder joint 108 (also herein termed shoulder flexion), by up to at least 45°, or by up to at least 90°, by up to at least 120°, or by up to at least 180°. In some embodiments, shoulder flexion is more than 180°. In some embodiments, shoulder flexion is up to 250°, or up to 300°. In an exemplary embodiment, shoulder flexion is about 200°.

In some embodiments, radius 116 is adapted to flex and extend from elbow joint 120 (also herein termed elbow flexion), by up to at least 45°, or by up to at least 90°, or by up to at least 180°. In some embodiments, elbow flexion is more than 180°. In some embodiments, elbow flexion is up to 250°, or up to 300°. In an exemplary embodiment, elbow flexion is approximately 200°.

Typically, during surgical procedures, articulated mechanical arm(s) 101 enter a body 160, via a body cavity or via an incision. Typically, torso(s) 102 extend through the entry point, and the remainder of arm(s) 101 are disposed within the body.

In some embodiments, an arm 101 within the body bends such that tool 124 thereof faces toward torso 102, as shown in FIG. 1A. This type of bending is termed “retroflex bending” herein. It is to be appreciated that arm 101 is considered to be bent in retroflex also if a vector of the direction in which the arm is pointing has a component that faces toward torso 102, but the tool 124 is not facing in a direction parallel to torso 102.

In some embodiments, arm 101 within the body bends such that tool 124 thereof faces away from torso 102, as shown in FIGS. 1B and 1C. This type of bending is termed “antigrade bending” herein. It is to be appreciated that arm 101 is considered to be bent in antigrade also if a vector of the direction in which the arm is pointing has a component that faces away from torso 102, but the tool 124 is not facing in a direction parallel to torso 102.

FIG. 1A schematically illustrates two types of retroflex bending that can be accomplished by arms 101. In the version indicated as arm 101a, the flexible portions of the arm, and particularly tool 124a, bends upward to a tool line 130 relative to torso 102 extending through the entry point and relative to a bed 135 on which the body is located. One can imagine the horizon of the arm 101 as being a plane extending through the incision or entry point into the body and through torso 102 of the arm, indicated in FIG. 1A by reference numeral 140. In this case, tool line 130 is disposed “above the horizon” relative to bed 135, which functions as the reference surface. As such, tool 124a of arm 101a is said to be disposed “above the horizon”, and arm 101a is said to “retroflex above the horizon”.

Conversely, in the version indicated as arm 101b, the flexible portions of the arm, and particularly tool 124b, bends downward to a tool line 150 relative to horizon 140 and relative to bed 135. In this case, tool line 150 is disposed “below the horizon” relative to the reference surface of bed 135. As such, tool 124b of arm 101b is said to be disposed “below the horizon”, and arm 101b is said to “retroflex below the horizon”.

FIGS. 1B and 1C schematically illustrates two types of antigrade bending that can be accomplished by arms 101. As seen in FIG. 1B, the flexible portions of arm 101, and particularly tool 124, bends at upward to a tool line 160 relative to torso 102 extending through the entry point and relative to bed 135. In FIG. 1B, the horizon is indicated by reference numeral 165. In this case, tool line 160 is disposed “above the horizon” relative to the reference surface of bed 135. As such, tool 124 of arm 101 is said to be disposed “above the horizon”, and arm 101 is said to “antigrade above the horizon”. The bending of the arm shown in FIG. 1B is also known as an S-shaped bend, above the horizon.

Conversely, as seen in FIG. 1C, the flexible portions of arm 101, and particularly tool 124, bends at upward to a tool line 170 relative to torso 102 extending through the entry point and relative to bed 135. In FIG. 1C, the horizon is indicated by reference numeral 175. In this case, tool line 170 is disposed “below the horizon” relative to the reference surface of bed 135. As such, tool 124 of arm 101 is said to be disposed “below the horizon”, and arm 101 is said to “antigrade below the horizon”. The bending of the arm shown in FIG. 1C is also known as an S-shaped bend, below the horizon.

In some embodiments, the device is stand-alone and, for example, does not require support of a user or of another support structure. In some embodiments, one or more portion of the device is at least partially supported by a support structure. In some embodiments, the user does not directly interact with the device. In some embodiments, movement of the device is substantially automated.

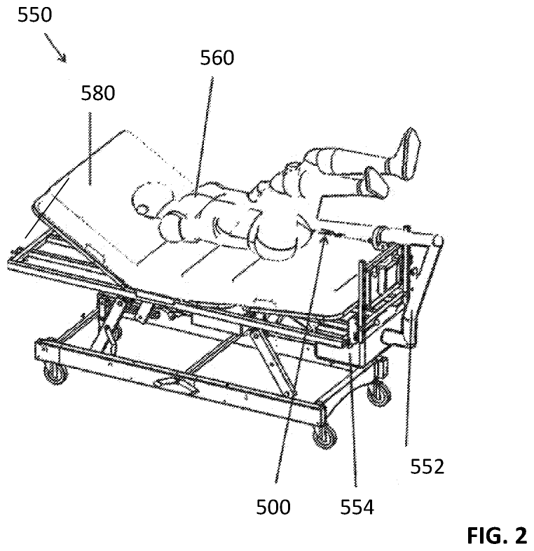

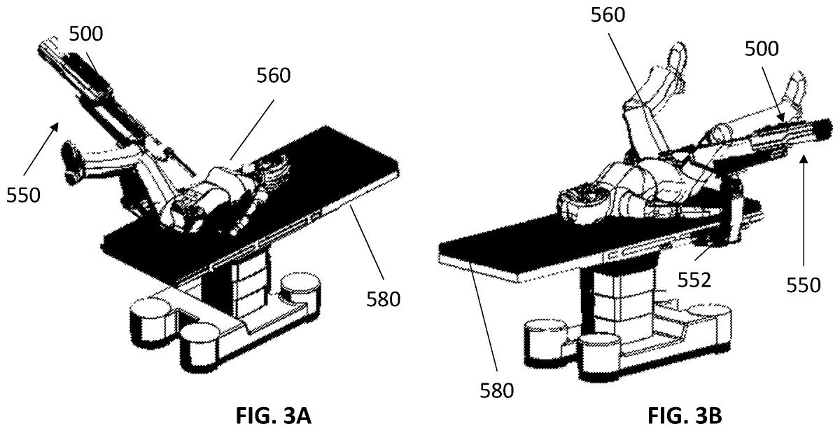

FIG. 2 is a simplified schematic view of a system 550 where a device 500 is held by a support structure 552 in a position suitable for trans-Pfannenstiel surgery, according to some embodiments of the invention. FIGS. 3A and 3B are simplified schematic views of system 550 where device 500 is held by the support structure 552 in a position suitable for trans-umbilical surgery, according to some embodiments of the invention.

In some embodiments, device 500 is coupled to a bed 580. In some embodiments, a patient 560 lies on bed 580 for surgical procedures using device 500. In some embodiments, one or more component of the device, for example one or more parts of the device control (e.g. motors) are located beneath the bed. In some embodiments, support structure 552 connects device 500 to bed 580. Optionally, other components, for example transformers, connectivity to other components e.g. the display, are located in a housing 554, which may be under the bed.

In an exemplary embodiment, a main motor unit for control of movement of the device is located in the housing, where for example, in some embodiments, torque transfer element(s) transfer torque from motor(s) within the housing to device 500 and/or elongated elements for effecting flexion of device joints are coupled to motors within the housing.

In some embodiments, control of movement of the device above the bed, using a motor unit underneath the bed is via an orientation controller, for example using a parallelogram linkage, e.g. as described in International Patent Application Publication No. WO2011/036626 which is herein incorporated by reference into the specification in its entirety.

One or more components being located beneath the bed (e.g. inside the housing), may reduce a footprint of the system in an operating room. Additionally, this arrangement may potentially improve access to the patient (e.g. in an emergency situation).

The device being coupled to a bed may enable moving and/or changing of an angle of the bed, for example, during surgery, while the device remains in the same position relative to the bed and/or to the patient. In some embodiments, a device position with respect to the patient and/or the bed is adjustable, for example, before treatment with the device and/or during surgery.

Optionally, in some embodiments, support 552 is adapted to move device 500 into position for surgery. In some embodiments, support 552 is adapted to move device 500 into a desired position for insertion of arms of the device into patient 560. In some embodiments, support 552 is adapted to move the device vertically, horizontally, and/or laterally. In some embodiments, support 552 is adapted to insert device 500 into patient 1160 and/or to withdraw device 500 from the patient.

In the embodiments illustrated in FIG. 2, support 552 and device 500 are arranged for entry of the device into the patient's body via a Pfannenstiel incision. However, the support and device of FIG. 2 may similarly be used for conducting the surgery via a sub-Pfannenstiel incision.

Specifically, in this arrangement, support structure 552 is located at the foot end of bed 580, such that device 500 extends over the pubic area of the patient toward the bikini line thereof. In FIG. 2, patient 560 is illustrated in a suitable position for insertion of device 500 via a small Pfannenstiel incision, with the patient's legs being elevated and apart (e.g. held by stirrups which are not shown). In some embodiments, when the device 500 enters the body via a Pfannenstiel incision, a camera functionally associated with the device enters the body via a small umbilical incision, as explained in further detail hereinbelow.

In the embodiments illustrated in FIGS. 3A and 3B, support 552 and device 500 are arranged for entry of the device into the patient's body via a small umbilical incision. Specifically, in this arrangement, support structure 552 is located along the lateral edge of bed 580, approximately at the center of the longitudinal axis thereof, such that device 500 extends over the umbilical area of the patient. In FIGS. 3A and 3B, patient 560 is illustrated with the patient's legs being elevated and apart (e.g. held by stirrups which are not shown). However, for trans-umbilical access of the device 500, the patient may also assume other positions, such as lying fully supine on bed 580. In some embodiments, when the device 500 enters the body via an umbilical incision, a camera functionally associated with the device also enters the body via the umbilical incision.

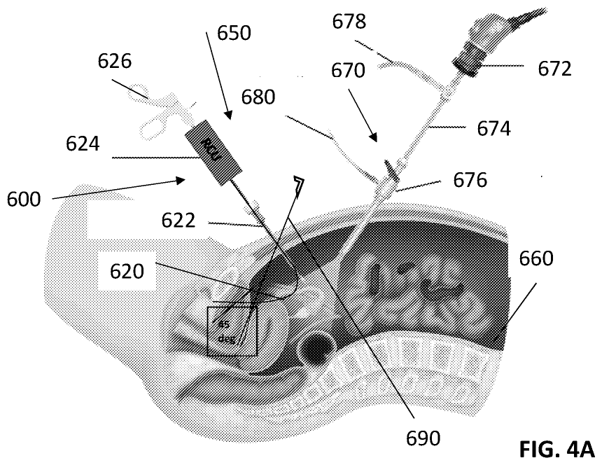

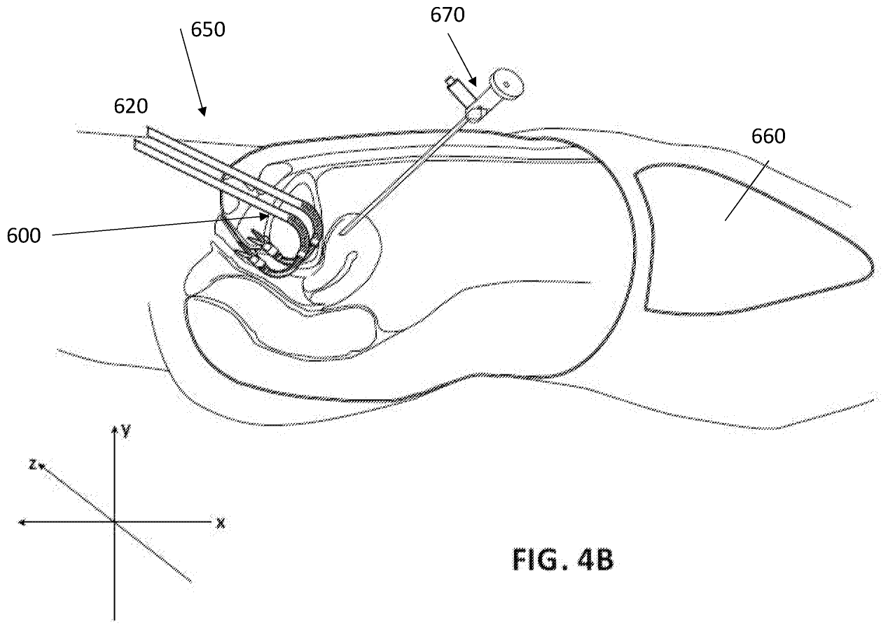

FIGS. 4A and 4B are schematic views of a position of a system 650 including a device 600, as described hereinabove with respect to FIGS. 1A to 1C, while conducting surgery for treatment of an inguinal hernia, via a small Pfannenstiel incision, according to some embodiments. However, in some embodiments, the surgery may similarly be conducted via a sub-Pfannenstiel incision.

As seen, the system 650 includes device 600 which enters a body 660 of a patient via the Pfannenstiel incision, and an imaging assembly 670 which enters the body 660 via an umbilical incision.

As seen, device 600 includes one or more articulated mechanical arms 620, which pass through a first trocar 622 positioned within the Pfannenstiel incision. Articulated mechanical arms(s) 620 may be controlled by a remote-control unit (RCU) 624 disposed outside the body of the patient. In some embodiments, mechanical arm(s) 620 may further be associated with laparoscopic scissors 626. As discussed hereinabove with respect to FIGS. 1A to 1C, the mechanical arm includes segments connected by joints, which are independently rotatable about a longitudinal axis of the segments and are independently bendable in a single bending plane.

Imaging assembly 670 includes a camera 672 disposed on a laparoscope 674, which enters the body via a second trocar 676 positioned within the umbilical incision. In some embodiments, imaging assembly 670 may further include a light source 678 and/or a carbon dioxide insufflation assembly 680, both functionally associated with laparoscope 674, for use during the surgical procedure.

For comparison, FIG. 4A additionally shows what would happen if one were to use a prior art “stick-arm” 690, as described in the prior art, to repair an inguinal hernia via a Pfannenstiel incision. As seen, prior art arm 690 would be inserted via the Pfannenstiel incision, and would be directed, as much as possible given the anatomy, toward the inguinal triangle. However, because the prior art arm 690 has a maximal bending range of 45 degrees (or less), it would be mechanically impossible to bring the prior art arm 690 through the Pfannenstiel incision, so that the arm reaches the inguinal triangle. The angular difference is simply too great to be covered using an arm with such a limited bending range.

By contrast, mechanical arm 620 according to embodiments of the invention bends around the pubic bone of patient 660, to be able to reach the inguinal hernia for treatment thereof. For treatment of an inguinal hernia, mechanical arm 620 must retroflex below the horizon, at an angle greater than 90 degrees (as explained hereinabove with respect to FIG. 1A), to facilitate surgery within the inguinal triangle (e.g. as the surgical zone), as clearly seen in FIGS. 4A and 4B. In some embodiments, the bending of arm 620 is such that the hernia is contacted from an access direction, the access direction being different by at least 90 degrees from a direction in which arm 620 enters the incision.

As explained in further detail hereinbelow, for treatment of a hernia, displaced tissue may be returned to its intended place, and the site of the hernia may be covered with a mesh. In some embodiments, the mesh may be inserted via the Pfannenstiel incision, as explained in further detail hereinbelow.

In some embodiments, multiple mechanical arms 620 may be inserted via the incision to treat the hernia. For example, this may be helpful when the hernia must be treated from multiple different angles, such as holding the mesh from different angles. As another example, multiple different tools may be used at the ends of the different mechanical arms.

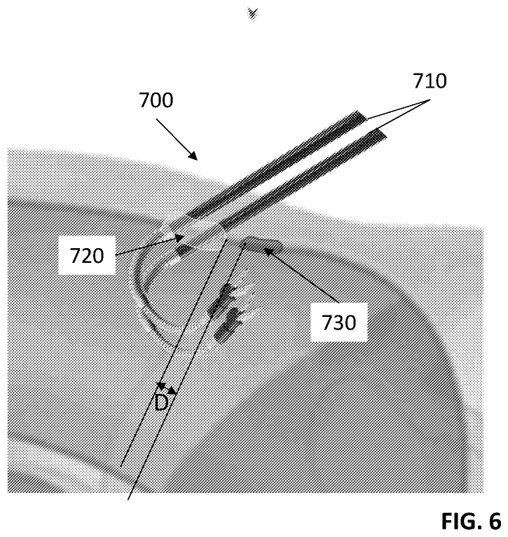

As explained in further detail hereinbelow with respect to FIG. 6, the surgical zone may be very close to the incision, such that a distance therebetween, or between the surgical zone and the first trocar 622, is less than 10 cm. In some embodiments, a ratio between the length of the mechanical arm 620 disposed within the body (i.e. a sum of the lengths of longitudinal axes of segments of the arm disposed within the body) and the distance between the incision/trocar and the surgical zone is at least 2:1, at least 3:1, at least 4:1, at least 5:1, at least 8:1, or at least 10:1. The distance may be measured along a body wall, such as along a pubic wall, and may be measured from the center of the incision to the center of the surgical zone, or from the edge of the incision to the edge of the surgical zone.

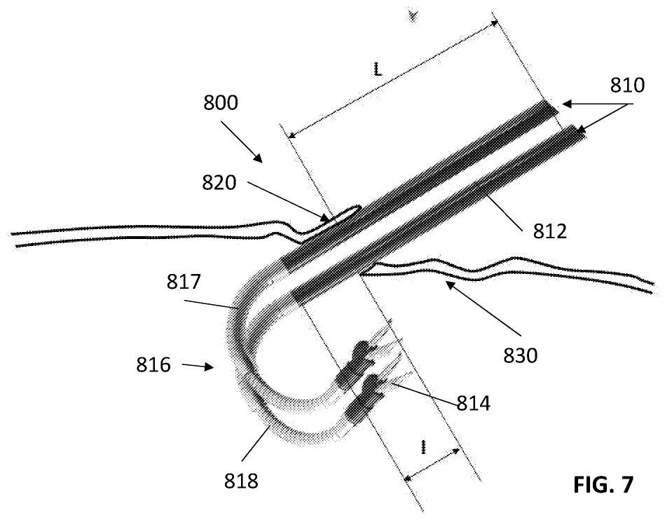

As explained in further detail hereinbelow with respect to FIG. 7, at least a majority of the linear portion of the mechanical arm(s) (torso 102 shown in FIG. 1A), and in some embodiments the entirety of the linear portion, is maintained outside the body during the hernia repair surgery. such that a distance therebetween, or between the surgical zone and the first trocar 622, is less than 10 cm.

In some embodiments, the inguinal hernia may be an internal inguinal hernia, where tissue is caught within the inguinal (Hasselbach) triangle. In some embodiments, the inguinal hernia may be an external inguinal hernia, where tissue (e.g. of the intestine) extends through, and out of, the inguinal triangle.

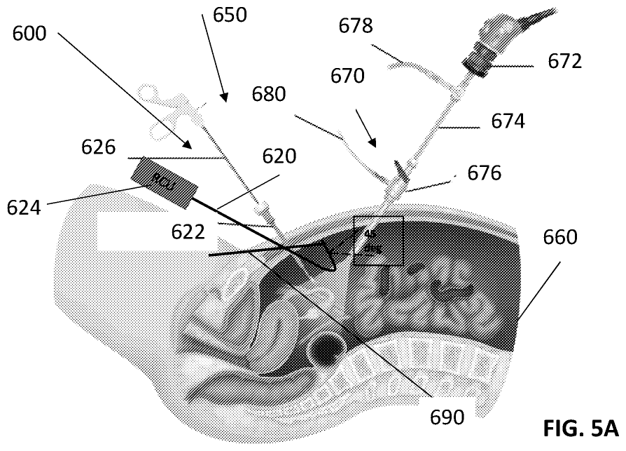

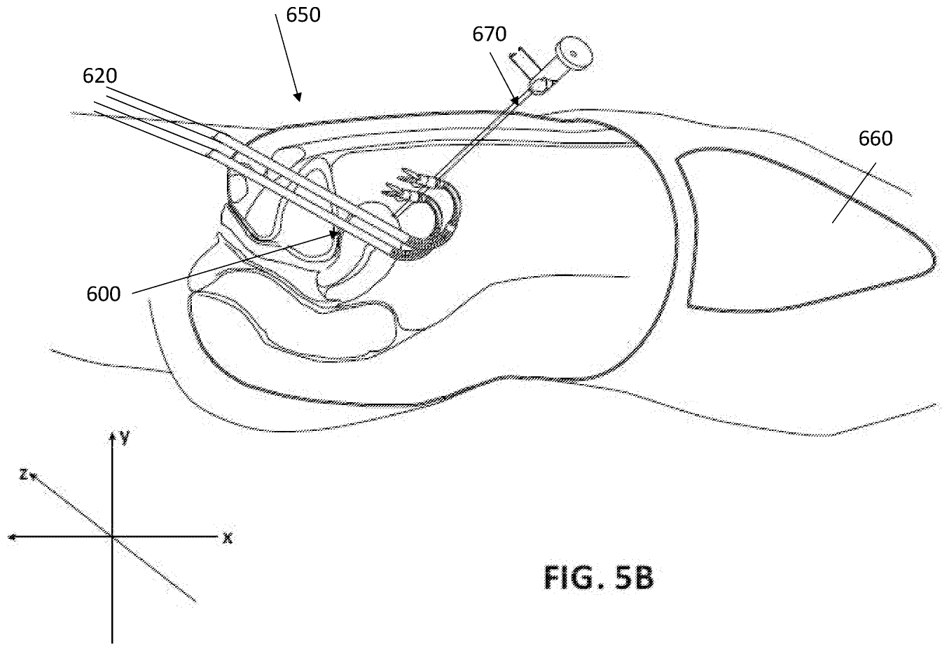

Reference is now made to FIGS. 5A and 5B, which are schematic views of a position of device 600 and of system 650 while conducting surgery for treatment of a ventral hernia, via a small Pfannenstiel incision, according to some embodiments. However, in some embodiments, the surgery may similarly be conducted via a sub-Pfannenstiel incision.

As seen, the ventral hernia is disposed along the abdominal wall of the patient's body, between the Pfannenstiel incision and the umbilicus. The system and device illustrated in FIGS. 5A and 5B are substantially identical to those shown in FIGS. 4A and 4B, and, for brevity, the description thereof is not repeated herein.

For comparison, FIG. 5A additionally shows what would happen if one were to use a prior art “stick-arm” 690, as described in the prior art, to repair a ventral hernia via a Pfannenstiel incision. As seen, prior art arm 690 would be inserted via the Pfannenstiel incision, and would be directed, as much as possible given the anatomy, toward the ventral hernia. However, because the prior art arm 690 has a maximal bending range of 45 degrees (or less), it would be mechanically impossible to bring the prior art arm 690 through the Pfannenstiel incision, so that the arm reaches a ventral hernia disposed on the abdominal wall beneath the umbilicus. The angular difference is simply too great to be covered using an arm with such a limited bending range.

By contrast, mechanical arm 620 according to embodiments of the invention is able to reach the ventral hernia for treatment thereof. For treatment of a ventral hernia along the abdominal wall, mechanical arm 620 must retroflex above the horizon, at an angle greater than 90 degrees, as explained hereinabove with respect to FIG. 1A and as clearly seen in FIGS. 5A and 5B. In some embodiments, the bending of arm 620 is such that the hernia is contacted from an access direction, the access direction being different by at least 90 degrees from a direction in which arm 620 enters the incision.

The treatment of a ventral hernia is substantially similar to the treatment of an inguinal hernia, as described herein, and may include use of multiple mechanical arms, placement of mesh, and work in a surgical zone which is very close to the incision.

Reference is now made to FIG. 6, which is a schematic view of a device 700 of system 750 while conducting abdominal surgery, according to some embodiments. Device 700 includes one or more mechanical arms 710, as described hereinabove with respect to FIGS. 1A to 1C.

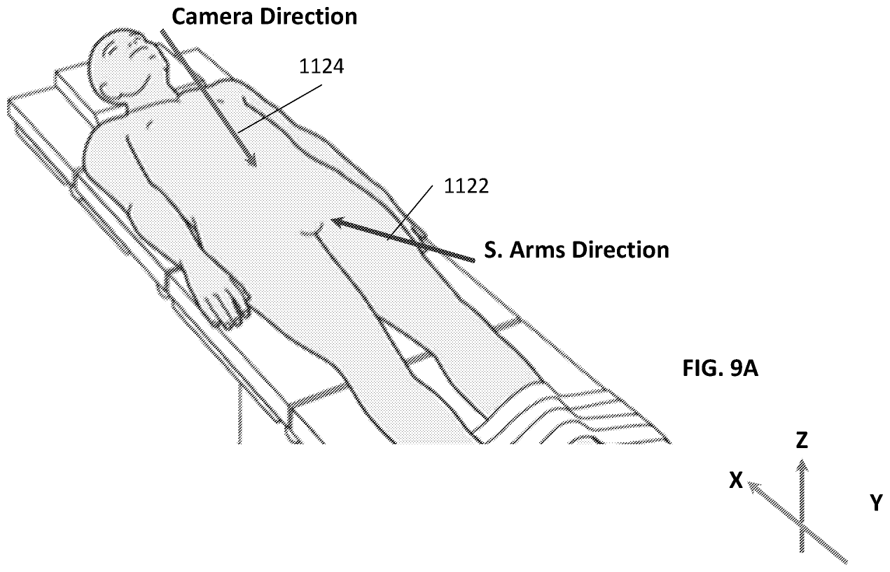

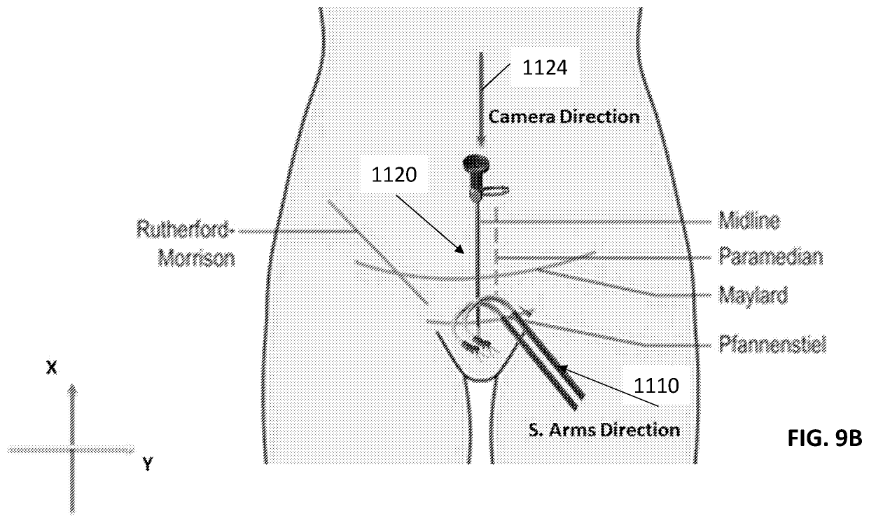

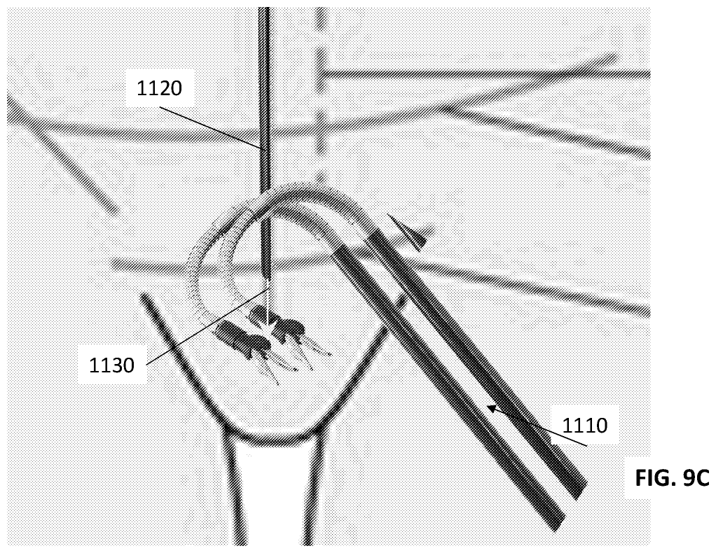

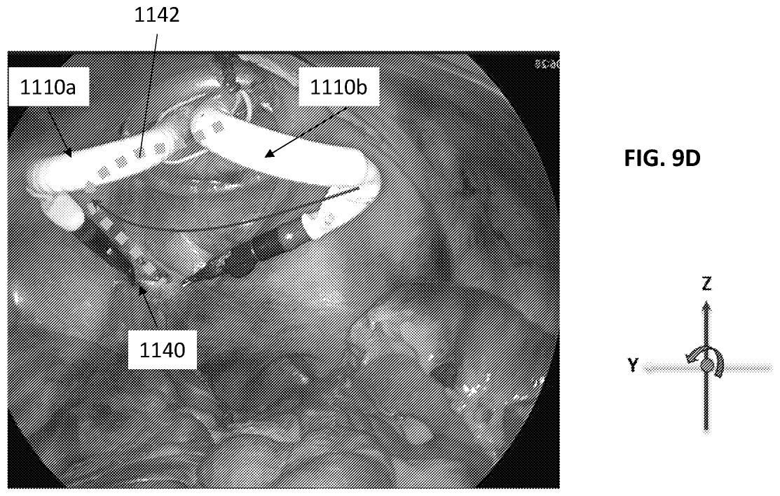

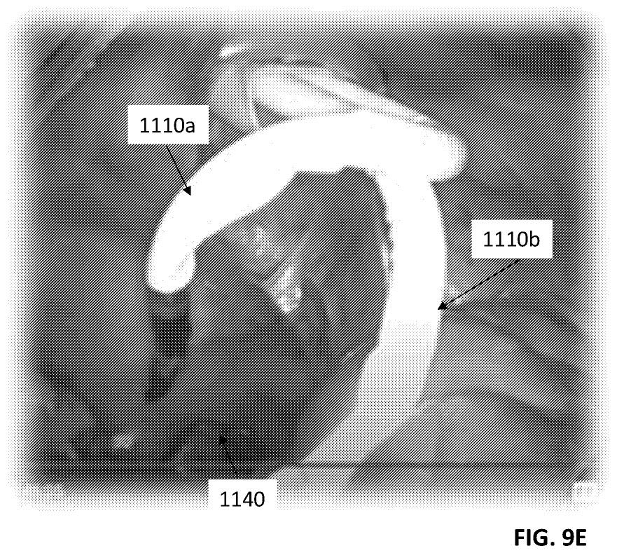

As discussed hereinabove with respect to FIGS. 4A to 5B, mechanical arms 710 enter the body through an incision zone 720, and conduct surgery in a surgical zone 730. As seen clearly in FIG. 9, there may be a very small distance D between the incision zone 720 and the surgical zone 730. For example, distance D may be less than 10 cm, less than 8 cm, less than 5 cm, less than 4 cm, less than 3 cm, less than 2 cm, or less than 1 cm.

In some embodiments, the distance D may be measured between the center of the incision and the center of the surgical zone. In some embodiments, the distance D may be a shortest distance measured between any point in the incision and any point of the surgical zone. Distance D may be measured along a wall of the body, such as along the abdominal wall, or may be measured in the shortest route possible, including through the abdominal cavity. In some embodiments, the distance D may be measured between a base of a trocar placed within the incision, and a point of the surgical zone closest to the trocar.

In some embodiments, a ratio between the length of arm 710 disposed within the body, and distance D, is at least 1.5:1, at least 2:1, at least 3:1, at least 4:1, at least 5:1, at least 8:1, or at least 10:1. The length of arm 710, disposed within the body, may be measured as a sum of the longitudinal axis lengths of portions of the arm disposed within the body. The sum typically includes the longitudinal lengths of the joints, but need not necessarily.

FIG. 7 is a schematic view of a device 800 of a system 850 while conducting abdominal surgery, according to some embodiments. Device 800 includes one or more mechanical arms 810, as described hereinabove with respect to FIGS. 1A to 1C. Each of mechanical arms 810 includes a linear portion 812 (similar to torso 102 in FIG. 1A), a tool 814 disposed at an end of the mechanical arm, and a flexible portion 816 extending between an end of linear portion 812 and tool 814. Flexible portion 816 typically includes a first joint 817 (similar to shoulder joints 108 in FIG. 1A) and a second joint 818 (similar to elbow joints 120 in FIG. 1A).

As discussed hereinabove with respect to FIGS. 4A to 5B, mechanical arms 810 enter the body through a small incision 820, which may be a Pfannenstiel incision, a sub-Pfannenstiel incision, or an umbilical incision, to conduct surgery in a surgical zone 830.

For the purpose of abdominal surgery, such as surgery for treating a hernia, the majority of the linear portions 812 of the arms is disposed outside of the body, and only a minority of the linear portions of the arms enters the body via the incision. In some embodiments, the entirety of the linear portions 812 may remain outside the body, such that the proximal-most point of arms 810 entering the body is the first joint 817.

In some embodiments, a ratio between a length of the section of linear portions 812 disposed within the body, indicated by ‘l’ in FIG. 7, and a length of the section of linear portions 812 disposed outside the body, indicated by L in FIG. 7, is at least 1:3, at least 1:5, at least 1:8, or at least 1:10.

In some embodiments, once the arms 810 are inserted through incision 820 into the body, if the remote-control unit outside the body remains in a fixed position, also linear arm portions 812 remain immobile relative to the incision and to the tissue surrounding the incision. In some embodiments, a ratio between a length of the movable portion of the arms 810 disposed within the body, (i.e. the length of the entirety of flexible portion 816), and a length of the portion of the arms 810 which is in a fixed position within the body (i.e. the length ‘l’ of the section of linear portions 812 disposed within the body) is at least 3:1, at least 5:1, at least 8:1, or at least 10:1.

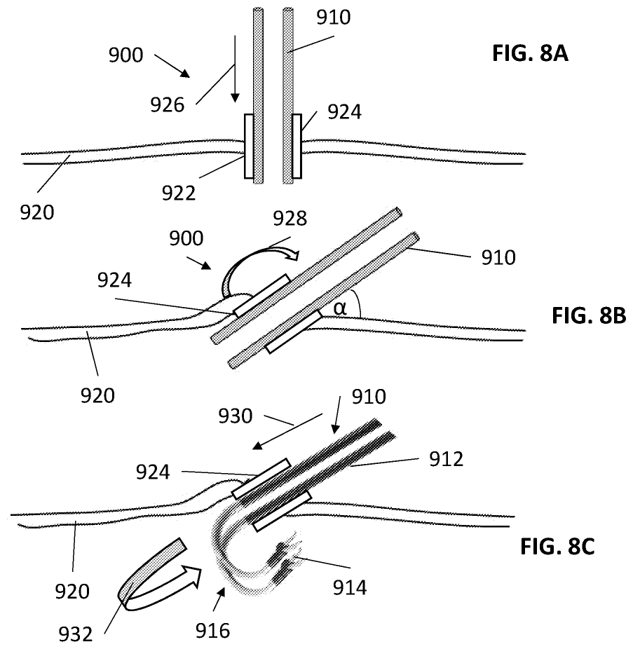

FIGS. 8A, 8B, and 8C are schematic views of steps of insertion of a device 900 of a system 950 into the abdomen while conducting surgery, according to some embodiments. Device 900 includes one or more mechanical arms 910, as described hereinabove with respect to FIGS. 1A to 1C. Each of mechanical arms 910 includes a linear portion 912 (similar to torso 102 in FIG. 1A), a tool 914 disposed at an end of the mechanical arm, and a flexible portion 916 extending between an end of linear portion 912 and tool 914. Flexible portion 916 typically includes a first joint (similar to shoulder joint 108 in FIG. 1A) and a second joint (similar to elbow joint 120 in FIG. 1A).

As seen in FIG. 8A, for conducting of surgery in abdominal tissue 920 having an incision 922 therein, a trocar 924, which may also be termed a cannula or port, may be inserted into the incision in a first angular orientation, here shown as being substantially vertical to tissue 920. Arms 910 may be inserted into the body, to a first extent, via incision 922 and trocar 924, in the first orientation, in the direction of arrow 926. Typically, tools 914, and some of flexible portions 916 will be inserted through trocar 924 at this initial entry stage. In some embodiments, the entry of arms 910 into the body at the angular orientation of FIG. 8A is limited by the presence of anatomical structures or constructs which obstruct the path for further insertion of arms 910. For example, when incision 922 is a Pfannenstiel incision into the abdomen, the pubic bone and/or the uterus may obstruct the arms from entering further into the body in the direction of arrow 926.

Consequently, as seen in FIG. 8B, trocar 924 together with arms 910 are pivoted, in the direction of arrow 928, such that the angular orientation between the longitudinal axes of trocar 924 and arms 910, relative to tissue 920, changes to be a second angular orientation. In the illustrated embodiment, in the second angular orientation an acute angle α is formed between tissue 920 and the longitudinal axis of arms 910. It is to be appreciated that during the pivoting shown in FIG. 8B, the longitudinal axes of arms 910 and of trocar 924 remain substantially parallel to each other, and there is minimal, or no, pivoting of arms 910 relative to the trocar.

Subsequently, arms 910 continue to be inserted into the body via trocar 924 in its new orientation, in the direction of arrow 930. Once a sufficient length of arms 910 has entered the body, arms 910 may bend, for example to retroflex in the direction of arrow 932, as seen in FIG. 8C, in order to access a surgical zone 940.

It is to be appreciated that, following insertion of the arms 910 into the body to the required extent, the surgery continues with the trocar 924 in the position shown in FIGS. 8B and 8C, and movement required for conducting the surgery is carried out by bending and rotation of arms 910, or of segments thereof, in various directions and into various orientations.

FIGS. 9A, 9B, 9C, 9D, and 9E are schematic views of a system 1150 including a device 1100, and a camera 1120 during conducting abdominal surgery via a trans-Pfannenstiel incision, according to some embodiments. Device 1100 includes one or more mechanical arms 1110, as described hereinabove with respect to FIGS. 1A to 1C. As seen in FIG. 9A, during surgery via a trans-Pfannenstiel incision, arms 1110 enter the body via the trans-Pfannenstiel incision, in the direction of arrow 1122, while camera 1120 enters the body via a trans-umbilical incision, in the direction of arrow 1124.

As seen, in order to reduce occlusion of the image captured by camera 1120, for example by the arms 1110 bending in retroflex below the horizon and blocking the camera's line of sight, the direction at which the arms enter the body is angled relative to the direction in which the camera enters the body, and is not along the midline of the body. This aspect is shown more clearly in FIG. 9B, which illustrates an obtuse angle β formed between the direction in which arms 1110 enter the body, and the direction in which camera 1120 enters the body. FIG. 9B further shows the bending of arms 1110 below the horizon, to treat a pubic region.

FIG. 9C illustrates that despite the relative angular orientation of arms 1110 and camera 1120, the line of sight of the camera may be blocked by the arms, as indicated by arrow 1130. Furthermore, in some embodiments, the camera would be viewing the arms from the height of the arms, or from beneath the arms, which may be very disorienting to surgeons who are accustomed to viewing the surgical zone from above the tools of the arms.

One possible solution to prevent, or reduce, such blocking of the field of view of the camera is shown in FIGS. 9D and 9E. As seen, rather than having arms be disposed parallel to one another as shown in FIGS. 9B and 9C, the arms are bent to be on opposing sides of the surgical zone 1140, thus allowing visual access to the surgical zone. Specifically, in FIG. 9D, arm 1110a remains in its place as previously discussed, while arm 1110b is moved from being in its previous location, indicated by dashed line 1142, to the opposing side of surgical zone 1140. This forms a shape similar to that of “frog legs”, and defines a window, e.g. having a rhombus shaped cross section, allowing the camera to have an unobstructed line of sight to surgical zone 1140.

FIG. 9E shows the surgical procedure from the point of view of the camera, entering through a trans-umbilical incision. As seen, the camera sees the tools of arms 1110 from above, in the manner convenient for a surgeon, and surgical zone 1140 is unobstructed. In some embodiments, in order to ease the surgeon's orientation relative to arms 1110, the arms may be crossed over, as seen in FIG. 9E.

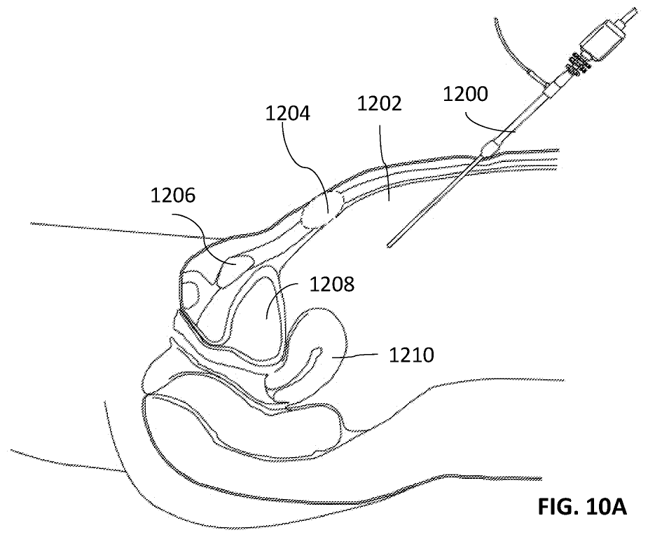

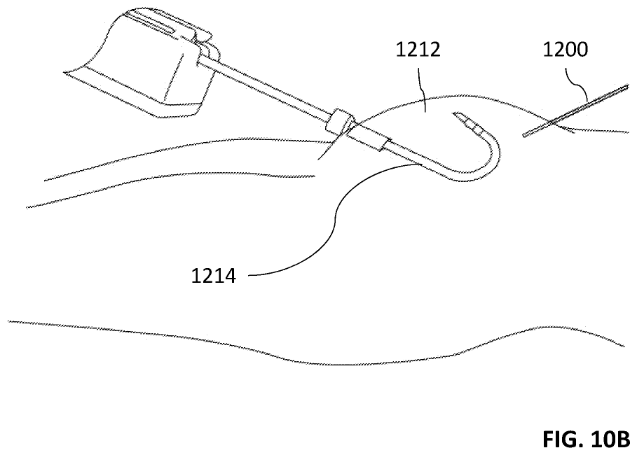

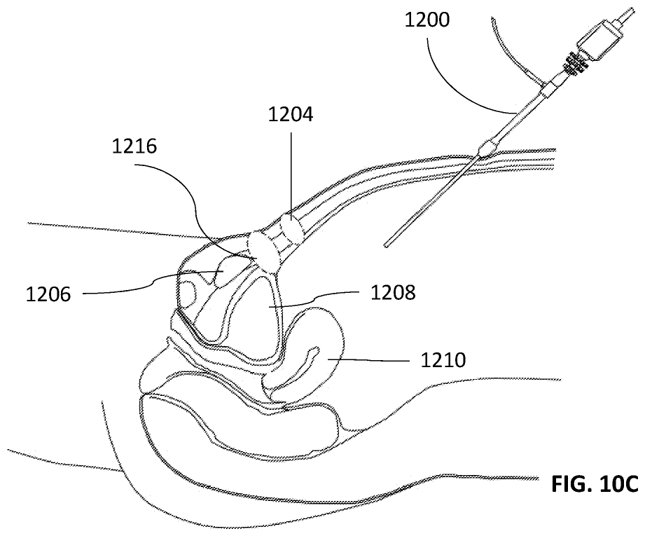

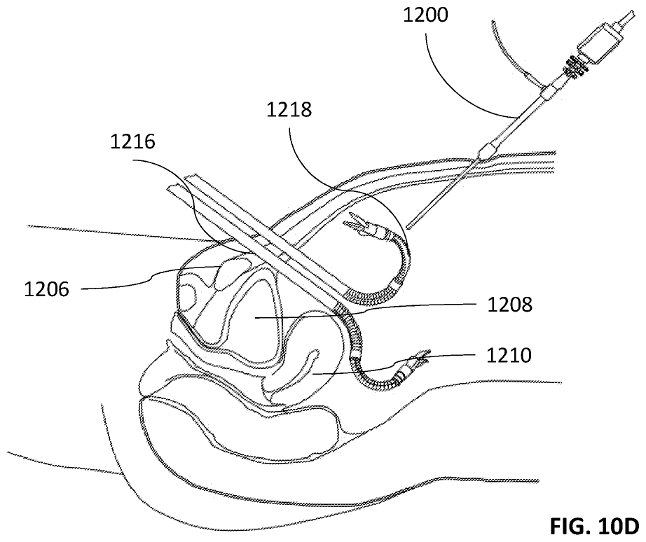

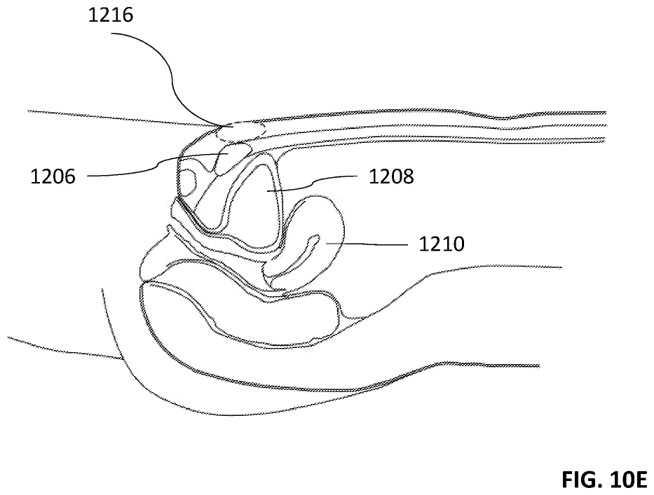

Reference is now made to FIGS. 10A, 10B, 10C, 10D, and 10E, which are schematic illustrations of methods for conducting abdominal surgery according to some embodiments.

As seen in FIG. 10A, a scope 1200, for example including a light source, a camera, and a source of gas, is inserted into a body 1202 of a subject via an umbilical incision, and is used to inflate the stomach of the subject. As described at length hereinabove, an incision zone 1204, in which an incision will be made for conducting the abdominal surgery, may be in the Pfannenstiel line, also known as the bikini line. The Pfannenstiel region is typically 2 fingers breadth (i.e. 2-5 cm) above the pubic bone 1206, and disposed higher than bladder 1208 and uterus 1210. FIG. 10B illustrates an abdominal surgery as described hereinabove, with stomach 1212 of the subject being inflated, scope 1200 entering the body via an umbilical incision, and articulated mechanical arms 1214 entering the body via a Pfannenstiel incision.

FIG. 10C is similar to FIG. 10A, and includes a different incision zone 1216, in a sub-Pfannenstiel region. As seen, different incision zone 1216 is in close proximity to the pubic bone 1206, for example within 1 cm from the pubic bone. Second incision zone 1216 remains above bladder 1208, but often is disposed below the highest point of uterus 1210, as shown. To be clear, when an incision is made in the sub-Pfannenstiel region, that incision replaces the Pfannenstiel incision described hereinabove.

FIG. 10D illustrates an abdominal surgery, with stomach 1212 of the subject being inflated, scope 1200 entering the body via an umbilical incision, and articulated mechanical arms 1218 entering the body via a sub-Pfannenstiel incision in second incision zone 1216. As seen, the articulated mechanical arms 1218 enter the body very close to the pubic bone 1206, and may almost lean on the pubic bone. In some embodiments, and as shown, all articulation of the mechanical arms is within the body, such that at least all the portions of the mechanical arms disposed outside the body remain linear.

In some embodiments, the surgery of FIG. 10D may be conducted using any of the systems, tools, or methods described hereinabove, including conducting the surgery via a trocar or another access kit, modifying the angle of the mechanical arms relative to the stomach following partial insertion of the mechanical arms into the incision, and use of various lengths, distances, and ratios, as described at length hereinabove.

It is to be appreciated that, although FIG. 10D illustrates the abdominal surgery via the sub-Pfannenstiel incision being carried out using articulated arms substantially as described hereinabove, the sub-Pfannenstiel incision can also be suitable for conducting surgery using straight stick mechanical arms, which include a linear portion terminating at a tool and do not have the ability to be articulated within the body.

FIG. 10E illustrates the body of FIGS. 10C and 10D, following the surgery, after stomach 1212 is no longer inflated. As seen, second incision zone 1216 has moved and is now located over the pubic bone 1206 because of the change in the distention level of stomach 1212. As such, the incisions (and resulting scar tissue) are not located over an abdominal section of the patient, following the surgery of FIG. 10D, significantly reducing the risk of an incisional hernia, even relative to an incision in the Pfannenstiel region which is considered a safer location for making abdominal incisions, as described hereinabove. In addition, the scar tissue being located lower on the patient body also provides certain aesthetic benefits as it is easier to conceal.

It is to be appreciated that any type of abdominal surgery may be conducted via a sub-Pfannenstiel incision as shown in FIGS. 10C to 10E, including, for example, surgeries for mending hernias as described hereinabove, hysterectomies, colectomies, appendectomies, Nissen fundoplication surgeries, cholecystectomies, sleeve gastrectomies, and gastric bypass surgeries.

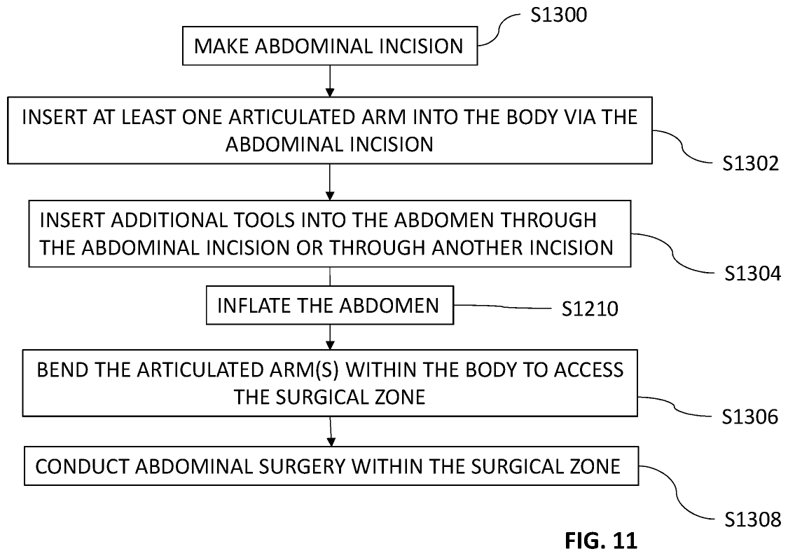

FIG. 11 is a flow chart of an exemplary method of conducting surgery within an abdominal surgical zone, according to some embodiments. In some embodiments, the system and/or device used to perform the treatment of hernia includes mechanical arms, as described herein. Alternatively, in some embodiments, other devices e.g. of the art which have the ability to bend within the body may be used.

At an initial step S1300, an abdominal incision is made in the body. In some embodiments, the incision may be an umbilical incision, a Pfannenstiel incision, or a sub-Pfannenstiel incision. In some embodiments, a length of the abdominal incision in in the range of 1 cm to 5 cm, 2 cm to 5 cm, 1 cm to 4 cm, 2 cm to 4 cm, 1 cm to 3 cm, 2 cm to 3 cm, or 1 cm to 2 cm.

At step S1302, at least one articulated, or jointed, mechanical arm of a device, as described herein, is inserted into the body via the abdominal incision. The arm includes a linear or rigid portion, at least a part of which (and in some embodiments the entirety of which) remains outside the body. As such, the flexible portion of the arm, as described hereinabove, is inserted into the body.

In some embodiments, the incision made at step S1300 is made using the device which enters the body at step S1302. In other embodiments, the incision may be made using a dedicated device, or a scalpel.

Optionally, in some embodiments, the device includes more than one articulated mechanical arm, e.g. as described elsewhere in this document. In an exemplary embodiment, the device includes two such arms. Optionally, in some embodiments, the device includes one or more additional tools.

In some embodiments, the device arm(s) are selected to be suitable for a particular patient and/or treatment. For example, in some embodiments, a user selects a device arm with a long humerus portion, when operating on an obese patient. For example, in some embodiments, a user selects a device arm with a short humerus portion, when operating on a child. In some embodiments, a length of one or more portion of the arm(s) may be changed during the surgical treatment.

In some embodiments, at step S1304, one or more laparoscopic tools or other devices are inserted through one or more additional incisions in the abdomen, to be used in the surgical treatment in addition to the device. For example, in a trans-Pfannenstiel or sub-Pfannenstiel surgical procedure, a camera may be inserted into the body via a trans-umbilical incision, in addition to the incision through which the device enters the body. It is to be appreciated however, that all the articulated arms participating in the surgery enter the body through a single incision.

At step S1306 one or more portions (e.g. limb, arm) of the device are bent within the body to access a surgical zone.

In some embodiments, the surgery is a hernia repair surgery, and the surgical zone is a site of the hernia. In some embodiments, in which the hernia is an inguinal hernia, the surgical zone may be in, or near, the inguinal triangle, also known as the Hasselbach triangle. In some embodiments, in which the hernia is a ventral hernia, the surgical zone may be along the abdominal wall.

In some embodiments, the surgery is a cholecystectomy, and the surgical zone is in or near the gall bladder, and/or along the abdominal wall.

In some embodiments, the surgery is colectomy, and the surgical zone is in or near a portion of the colon.

The direction in which the arm(s) bend is dependent on the spatial relationship between the location of the incision and the surgical zone. As discussed hereinabove with respect to FIGS. 4A to 5B, the arms may be bent to retroflex above the horizon, or below the horizon, as needed. In some embodiments, the arms may bend in another direction, or in an S-shape, for example as shown in FIGS. 1B and 1C.

In some embodiments, in which the device includes two or more arms, the arms of the device bend within a body in the same direction, for example approaching a target from the same direction, e.g. as illustrated by FIGS. 9B and 9C. In some embodiments, the arms bend substantially along the same trajectory, for one or more segment and/or joint, for example with or more joint and/or segment pair in contact and/or with the same orientation. In other embodiments, the arms may bend in different directions, or along different trajectories, for example in order to access the surgical zone from two different directions. An example of bending of the arms in two different directions is shown and discussed with respect to FIGS. 9D and 9E hereinabove.

Relating back to FIG. 1A, and specifically to arm 101 thereof—for the following discussion, angle A is defined as the angle between the longitudinal axis of first torso 102 and the longitudinal axis of first humerus 112; angle B is defined as the angle between the longitudinal axis of first humerus 112 and the longitudinal axis of first radius 116; and angle C is defined as the angle between the longitudinal axis of first radius 116 and the longitudinal axis of first hand tool 124. Angles A, B, and C may correspond, respectively, to angles of the shoulder, elbow, and wrist joints.

In some embodiments, bending of the device within the body refers to a sum of angles A, B, and C being at least 90°, at least 120°, at least 180°, at least 240°, or at least 360°, where at least two of angles A, B, and C are larger than zero. In some embodiments, one or more of angles A, B, or C is adjustable by up to 180°, for example with enabling bending of the device of up to 540°. In an exemplary embodiment, angles A and B are adjustable by up to 180° and angle C is adjustable by up to 90°. In some embodiments, one or more of angles A, B, and C may be three dimensionally adjustable, such that the angle may have a z-axis component, in addition to x- and y-axis components.

In some embodiments, bending of the device within the body refers to a sum of angles A, B, and C, for at least one mechanical arm, being at least 90°, at least 120°, at least 180° in the x-y plane and/or in the x-z plane and/or in the z-y plane, where at least two of angles A, B, and C are larger than zero, in the x-y plane and/or in the x-z plane and/or in the z-y plane.

In some embodiments, bending of the device within the body is such that an access direction to the surgical zone is at an angle of at least 90°, at least 120°, or at least 180° relative to a direction of insertion of the arms into the incision, in at least one dimension. In some embodiments, a difference between the insertion direction and the access direction is defined by an angle D=A+B+C.

In some embodiments, in which the number of segments in the mechanical arms of the device is distinct from that shown in FIGS. 1A to 1C (e.g. more segments or fewer segments), the sum of angles relates to the sum all relevant angles for that number of segments, which may be a greater number of angles or a smaller number of angles.

At step S1308, the device conducts the surgical procedure, within the surgical zone.

In some embodiments, in which the surgical procedure is repair of a hernia, the procedure includes covering the site of the hernia with a mesh. In some embodiments, repair of the hernia includes, prior to the covering, moving displaced tissue, causing the hernia, through a second tissue. For example, this may include moving tissue of the intestine through inguinal tissue or through tissue of the abdominal wall, back into the abdominal cavity.

In some embodiments, a first arm of the device is used to separate (e.g. by pushing) the hernia tissue from the surrounding tissue, and a second arm is used to apply the mesh. In some embodiments, the mesh is inserted into the body through the abdominal incision, e.g. while held in a gripping tool of the arm. In some embodiments, the device may include a tissue manipulator, which may be mounted onto an arm of the device, to move obstructing tissue out of the way, to reduce the risk of damaging the obstructing tissue during the surgical procedure.

In embodiments in which the surgical procedure is treatment of an inguinal hernia, the procedure takes place within the inguinal triangle, which has a planar area of approximately 10 cm², following the bending, and while the tools used for the surgical procedure enter the body from a single abdominal incision, as discussed herein.

In some embodiments, in which the surgical procedure is colectomy or cholecystectomy, the procedure may include separating or excising tissue to be removed from the body from surrounding tissue. In some embodiments, the removed tissue is moved through a second tissue.

Optionally, in some embodiments, the abdomen is inflated (e.g. with CO2) before treatment and/or surgery begins, at an optional step S1310. In some embodiments, the abdomen is inflated through a different incision than that used for insertion of the device, such as the incision through which the camera is inserted. I additional incision (not the incision for insertion of the device). In some embodiments, the abdomen is inflated through the incision used for insertion of the device.

Exemplary embodiments of the present invention are provided hereinbelow:

Embodiment 1. A method of repairing a hernia, the method comprising:
inserting an articulated mechanical limb into a body through an abdominal incision, where the articulated mechanical limb comprises:
   a linear portion, at least part of which extends outside the body, the linear portion being coupled to a mount disposed outside the body;
   at least one flexible portion coupled to the linear portion;
   and
   a tool coupled to the at least one flexible portion;
bending the articulated mechanical limb within the body at the at least one flexible portion so that the tool contacts the hernia, wherein that a sum of angles between adjacent effective segment longitudinal axes, in at least one three dimensionally oriented plane, is greater than 90°; and
repairing the hernia with the tool of the articulated mechanical limb, where the repairing comprises covering the site of the hernia with a mesh.

Embodiment 2. The method of embodiment 1, wherein the repairing of the hernia comprises moving displaced tissue of the hernia body through a second tissue.

Embodiment 3. The method of embodiment 1 or embodiment 2, wherein the hernia is an inguinal hernia, and the surgical zone is the inguinal triangle.

Embodiment 4. The method of embodiment 3, wherein the bending includes bending around the pubic bone of the body.

Embodiment 5. The method of embodiment 3 or embodiment 4, wherein the abdominal incision is outside of a surgical zone including the hernia, and the bending of the articulated mechanical limb is such that a length of the articulated mechanical limb within the body, measured as a sum of lengths of longitudinal axes of portions of the articulated mechanical limb within the body, is at least 1.5 times, at least 2 times, at least 3 times, or at least 4 times the distance between the abdominal incision and the inguinal hernia, where the distance is a shortest distance between the abdominal incision and the inguinal hernia when measured along the lower pelvic wall.

Embodiment 6. The method of embodiment 1 or embodiment 2, wherein the hernia is a ventral hernia, and the surgical zone is a surface of the abdominal wall.

Embodiment 7. The method of embodiment 6, wherein the bending comprises bending of the at least one flexible portion such that the tool is disposed above the horizon relative to the linear portion entering the abdominal incision.

Embodiment 8. The method of embodiment 6, wherein the bending comprises bending of the at least one flexible portion such that the tool is disposed in a tool plane, the tool plane being above an incision plane formed by the abdominal incision and the linear portion, relative to a reference surface.

Embodiment 9. The method of any one of embodiments 6 to 8, wherein the abdominal incision is outside of a surgical zone including the hernia, and the bending of the articulated mechanical limb is such that a length of the articulated mechanical limb within the body, measured as a sum of lengths of longitudinal axes of portions of the articulated mechanical limb within the body, is at least 1.5 times, at least 2 times, at least 3 times, or at least 4 times the distance between the abdominal incision and the ventral hernia, where the distance is a shortest distance between the abdominal incision and the ventral hernia when measured along the abdominal wall.

Embodiment 10. The method of any one of embodiments 1 to 9, wherein the abdominal incision is a Pfannenstiel incision or a sub-Pfannenstiel incision.

Embodiment 11. The method of any one of embodiments 1 to 9, wherein the abdominal incision is an umbilical incision.

Embodiment 12. The method of any one of embodiments 1 to 11, wherein a length of the abdominal incision is in the range of 1 cm to 5 cm, 2 cm to 5 cm, 1 cm to 4 cm, 2 cm to 4 cm, 1 cm to 3 cm, or 2 cm to 3 cm.

Embodiment 13. The method of any one of embodiments 1 to 12, wherein the bending of the articulated mechanical limb is to contact the hernia from an access direction where the access direction is different by at least 90° from a direction of entry of the articulated mechanical limb into the body.

Embodiment 14. The method of any one of embodiments 1 to 13, further comprising inserting the mesh into the body through the abdominal incision.

Embodiment 15. The method of any one of embodiments 1 to 14, wherein the at least one flexible portion of the articulated mechanical limb comprises a first flexible portion coupled to the linear portion and a second flexible portion coupled at one end thereof the first flexible portion and at an opposing end thereof to the tool.

Embodiment 16. The method of embodiment 15, wherein each of the first and second flexible portions is independently rotatable about its longitudinal axis, and is independently bendable in a single bending plane.

Embodiment 17. The method of any one of embodiments 15 to 16, wherein each of the first and second flexible portions is bendable in one rotational direction around each corresponding bending plane.

Embodiment 18. The method of any one of embodiments 1 to 17, wherein the repairing comprises holding a portion of body tissue away from the surgical zone.

Embodiment 19. The method of any one of embodiments 1 to 18, wherein the inserting comprises inserting a plurality of the articulated mechanical limbs into the body through the abdominal incision, and the repairing comprises repairing the hernia using the tools of the plurality of articulated mechanical limbs.

Embodiment 20. The method of any one of embodiments 1 to 19, further comprising inflating an abdominal cavity prior to, or during, the repairing.

Embodiment 21. The method of any one of embodiments 1 to 20, wherein the abdominal incision is outside of a surgical zone including the hernia, and a shortest distance between the abdominal incision and the surgical zone, measured along a wall of the body, is less than 10 cm, less than 8 cm, less than 5 cm, less than 4 cm, less than 3 cm, less than 2 cm, or less than 1 cm.

Embodiment 22. The method of any one of embodiments 1 to 20, wherein the inserting comprises inserting the articulated mechanical limb through a trocar located in the abdominal incision, the abdominal incision is outside of a surgical zone including the hernia, and a shortest distance between a base of the trocar and the surgical zone, measured along a wall of the body, is less than 10 cm, less than 8 cm, less than 5 cm, less than 4 cm, less than 3 cm, less than 2 cm, or less than 1 cm.

Embodiment 23. The method of any one of embodiments 1 to 22, wherein the inserting comprises inserting the at least one flexible portion into the body, while maintaining at least a majority of the linear portion outside of the body.

Embodiment 24. The method of any one of embodiments 1 to 22, wherein the inserting comprises inserting the at least one flexible portion into the body, while maintaining the entirety of the linear portion outside of the body.

Embodiment 25. The method of any one of embodiments 1 to 22, wherein a ratio between a length of the articulated mechanical limb disposed outside the body and a length of the articulated mechanical limb disposed within the body, measured as a sum of lengths of longitudinal axes of portions of the articulated mechanical limb within the body, is in the range of 1:2-1:4.

Embodiment 26. The method of any one of embodiments 1 to 22, wherein a ratio between a length of portions of the articulated mechanical limb disposed outside the body and a length of portions of the articulated mechanical limb disposed within the body, measured as a sum of lengths of longitudinal axes of portions of the articulated mechanical limb within the body, is at least 1:2 or at least 1:3.

Embodiment 27. The method of any one of embodiments 1 to 22, wherein a ratio between a length of the articulated mechanical limb, disposed within the body, which is stationary relative to the abdominal incision, and a length of the articulated mechanical limb disposed within the body which is movable relative to the abdominal incision, when the mount outside the body is stationary, is at least 1:5, at least 1:8, at least 1:10, or at least 1:15.

Embodiment 28. The method of any one of embodiments 1 to 27, wherein the inserting comprises, in order:
placing a trocar in the abdominal incision, while the trocar is in a first angular orientation relative to abdominal tissue including the incision;
inserting the articulated mechanical limb through the trocar to a first extent while the trocar is in the first angular orientation, such that a first segment of the articulated mechanical limb is disposed within the body;
changing an angular orientation of the trocar, together with the articulated mechanical limb, relative to the abdominal tissue; and
inserting the articulated mechanical limb through the trocar to a second extent while the trocar is in the second angular orientation, such that the first segment and an additional second segment of the articulated mechanical limb are disposed within the body.

Embodiment 29. The method of embodiment 28, wherein the first angular orientation is substantially perpendicular to the abdominal tissue, and the second angular orientation is at an acute angle relative to the abdominal tissue.

Embodiment 30. A method of carrying out abdominal surgery in a surgical zone of a body, the method comprising:
inserting two mechanical limbs into a body through a single abdominal incision, each of the mechanical limbs terminating in a tool, where at least one of the two mechanical limbs is an articulated mechanical limb, comprising:
   a linear portion, at least part of which extends outside the body, the linear portion being coupled to a mount disposed outside the body; and
   at least one flexible portion coupled to the linear portion,
   wherein the tool is coupled to the second flexible portion;
bringing the tools of the mechanical limbs to the surgical zone, wherein the bringing comprises bending the at least one articulated mechanical limb within the body at the at least one flexible portion such that the tool thereof enter the surgical zone, wherein a sum of angles between adjacent effective segment longitudinal axes, in at least one three dimensionally oriented plane, is greater than 90°; and
using the tools of the two mechanical limbs, conducting surgery within the surgical zone.

Embodiment 31. The method of embodiment 30, wherein the surgical zone has an area of at most 10 cm².

Embodiment 32. The method of embodiment 31, wherein the bending is carried out within a volume having a cross section greater than 10 cm².

Embodiment 33. The method of any one of embodiments 30 to 32, wherein the abdominal surgery comprises hernia surgery, and wherein the conducting of the surgery comprises repairing a hernia in the surgical zone using the tools of the mechanical limbs.

Embodiment 34. The method of embodiment 33, wherein the repairing comprises covering the site of the hernia with a mesh.

Embodiment 35. The method of embodiment 34, further comprising, inserting the mesh into the body through the abdominal incision.

Embodiment 36. The method of any one of embodiments 33 to 35, wherein the repairing of the hernia comprises moving displaced tissue of the hernia body through a second tissue.

Embodiment 37. The method of any one of embodiments 33 to 36, wherein the hernia is an inguinal hernia, and the surgical zone is the inguinal triangle.

Embodiment 38. The method of embodiment 37, wherein the bending includes bending around the pubic bone of the body.

Embodiment 39. The method of any one of embodiments 31 to 38, wherein the abdominal incision is outside of the surgical zone, and the bending of the at least one articulated mechanical limb is such that a length of the at least one articulated mechanical limb within the body, measured as a sum of lengths of longitudinal axes of portions of the at least one articulated mechanical limb within the body, is at least 1.5 times, at least 2 times, at least 3 times, or at least 4 times the distance between the abdominal incision and the surgical zone, where the distance is a shortest distance between the abdominal incision and the surgical zone when measured along a body wall.

Embodiment 40. The method of any one of embodiments 30 to 39, wherein the abdominal incision is a Pfannenstiel incision or a sub-Pfannenstiel incision.

Embodiment 41. The method of any one of embodiments 30 to 39, wherein the abdominal incision is an umbilical incision.

Embodiment 42. The method of any one of embodiments 30 to 41, wherein a length of the abdominal incision is in the range of 1 cm to 5 cm, 2 cm to 5 cm, 1 cm to 4 cm, 2 cm to 4 cm, 1 cm to 3 cm, or 2 cm to 3 cm.

Embodiment 43. The method of any one of embodiments 30 to 42, wherein the bending of the at least one articulated mechanical limb is to contact the surgical zone from an access direction where the access direction is different by at least 90° from a direction of entry of the at least one articulated mechanical limb into the body.

Embodiment 44. The method of any one of embodiments 30 to 43, wherein the at least one flexible portion of the articulated mechanical limb comprises a first flexible portion coupled to the linear portion and a second flexible portion coupled at one end thereof the first flexible portion and at an opposing end thereof to the tool.

Embodiment 45. The method of embodiment 45, wherein each of the first and second flexible portions is independently rotatable about its longitudinal axis, and is independently bendable in a single bending plane.

Embodiment 46. The method of embodiment 45 or embodiment 46, wherein each of the first and second flexible portions is bendable in one rotational direction around each corresponding bending plane.

Embodiment 47. The method of any one of embodiments 30 to 46, wherein the conducting surgery comprises holding a portion of body tissue away from the surgical zone.

Embodiment 48. The method of any one of embodiments 30 to 47, further comprising inflating an abdominal cavity prior to, or during, the conducting of the surgery.

Embodiment 49. The method of any one of embodiments 30 to 48, wherein the abdominal incision is outside of the surgical zone, and a shortest distance between the abdominal incision and the surgical zone, measured along a wall of the body, is less than 10 cm, less than 8 cm, less than 5 cm, less than 4 cm, less than 3 cm, less than 2 cm, or less than 1 cm.

Embodiment 50. The method of any one of embodiments 30 to 49, wherein the abdominal incision is outside of the surgical zone, the inserting comprises inserting the mechanical limbs through a trocar located in the abdominal incision, and wherein a shortest distance between a base of the trocar and the surgical zone, measured along a wall of the body, is less than 10 cm, less than 8 cm, less than 5 cm, less than 4 cm, less than 3 cm, less than 2 cm, or less than 1 cm.

Embodiment 51. The method of any one of embodiments 30 to 50, wherein the inserting comprises, in order:
placing a trocar in the abdominal incision, while the trocar is in a first angular orientation relative to abdominal tissue including the incision;
inserting the mechanical limbs through the trocar to a first extent while the trocar is in the first angular orientation, such that a first segment of the articulated mechanical limb is disposed within the body;
changing an angular orientation of the trocar, together with the mechanical limbs, relative to the abdominal tissue; and
inserting the mechanical limbs through the trocar to a second extent while the trocar is in the second angular orientation, such that the first segment and an additional second segment of the mechanical limbs are disposed within the body.

Embodiment 52. The method of embodiment 51, wherein the first angular orientation is substantially perpendicular to the abdominal tissue, and the second angular orientation is at an acute angle relative to the abdominal tissue.

Embodiment 53. The method of embodiment any one of embodiments 30 to 52, wherein the bending comprises bending of the at least one flexible portion such that the tool is disposed above the horizon relative to the linear portion entering the abdominal incision.

Embodiment 54. The method of embodiment any one of embodiments 30 to 52, wherein the bending comprises bending of the at least one flexible portion such that the tool is disposed in a tool plane, the tool plane being above an incision plane formed by the abdominal incision and the linear portion, relative to a reference surface.

Embodiment 55. A method of carrying out abdominal surgery, the method comprising:
inserting an articulated mechanical limb into a body through a Pfannenstiel incision or a sub-Pfannenstiel incision, where the articulated mechanical limb comprises:
   a linear portion, at least part of which extends outside the body, the linear portion being coupled to a mount disposed outside the body;
   at least one flexible portion coupled to the linear portion; and
   a tool coupled to the at least one flexible portion;
bending the mechanical limb within the body at the at least one flexible portion so that the tool thereof enters an abdominal surgical zone, the abdominal surgical zone being in a lower quadrant of the abdomen; and
using the tool, conducting surgery within the abdominal surgical zone.

Embodiment 56. The method of embodiment 55, wherein, during the bending, a sum of angles between adjacent effective segment longitudinal axes of the articulated mechanical limb, in at least one three dimensionally oriented plane, is greater than 90°.

Embodiment 57. The method of embodiment 55 or embodiment 56, further comprising, during the conducting of the surgery, moving the tools between at least two quadrants of the abdomen without moving the linear portion of the mechanical limb or the mount disposed outside the body.

Embodiment 58. The method of any one of embodiments 55 to 57, wherein the abdominal surgery comprises hernia surgery, and wherein the conducting of the surgery comprises repairing a hernia in the surgical zone using the tool of the articulated mechanical limb.

Embodiment 59. The method of embodiment 58, wherein the repairing comprises covering the site of the hernia with a mesh.

Embodiment 60. The method of embodiment 59, further comprising, inserting the mesh into the body through the incision.

Embodiment 61. The method of any one of embodiments 58 to 60, wherein the repairing of the hernia comprises moving displaced tissue of the hernia body through a second tissue.

Embodiment 62. The method of any one of embodiments 58 to 61, wherein the hernia is an inguinal hernia, and the surgical zone is the inguinal triangle.

Embodiment 63. The method of embodiment 62, wherein the bending includes bending around the pubic bone of the body.

Embodiment 64. The method of any one of embodiments 58 to 61, wherein the hernia is a ventral hernia, and the surgical zone is an abdominal wall in the lower quadrant of the abdomen.

Embodiment 65. The method of embodiment any one of embodiments 55 to 64, wherein the bending comprises bending of the at least one flexible portion such that the tool is disposed above the horizon relative to the linear portion entering the abdominal incision.

Embodiment 66. The method of embodiment any one of embodiments 55 to 65, wherein the bending comprises bending of the at least one flexible portion such that the tool is disposed in a tool plane, the tool plane being above an incision plane formed by the abdominal incision and the linear portion, relative to a reference surface.

Embodiment 67. The method of any one of embodiments 55 to 66, wherein the abdominal incision is outside of the abdominal surgical zone, the bending of the articulated mechanical limb is such that a length of the articulated mechanical limb within the body, measured as a sum of lengths of longitudinal axes of portions of the articulated mechanical limb within the body, is at least 1.5 times, at least 2 times, at least 3 times, or at least 4 times the distance between the incision and the surgical zone, where the distance is a shortest distance between the incision and the surgical zone when measured along a body wall.

Embodiment 68. The method of any one of embodiments 55 to 67, wherein a length of the incision is in the range of 1 cm to 5 cm, 2 cm to 5 cm, 1 cm to 4 cm, 2 cm to 4 cm, 1 cm to 3 cm, or 2 cm to 3 cm.

Embodiment 69. The method of any one of embodiments 55 to 68, wherein the bending of the articulated mechanical limb is to contact the surgical zone from an access direction where the access direction is different by at least 90° from a direction of entry of the articulated mechanical limb into the body.

Embodiment 70. The method of any one of embodiments 55 to 69, wherein the at least one flexible portion of the articulated mechanical limb comprises a first flexible portion coupled to the linear portion and a second flexible portion coupled at one end thereof the first flexible portion and at an opposing end thereof to the tool.

Embodiment 71. The method of embodiment 70, wherein each of the first and second flexible portions is independently rotatable about its longitudinal axis, and is independently bendable in a single bending plane.

Embodiment 72. The method of embodiment 70 or embodiment 71, wherein each of the first and second flexible portions is bendable in one rotational direction around each corresponding bending plane.

Embodiment 73. The method of any one of embodiments 55 to 72, wherein the conducting surgery comprises holding a portion of body tissue away from the surgical zone.

Embodiment 74. The method of any one of embodiments 55 to 73, further comprising inflating an abdominal cavity prior to, or during, the conducting of the surgery.

Embodiment 75. The method of any one of embodiments 55 to 74, wherein the abdominal incision is outside of the surgical zone, and a shortest distance between the incision and the surgical zone, measured along a wall of the body, is less than 10 cm, less than 8 cm, less than 5 cm, less than 4 cm, less than 3 cm, less than 2 cm, or less than 1 cm.

Embodiment 76. The method of any one of embodiments 55 to 74, wherein the abdominal incision is outside of the surgical zone, the inserting comprises inserting the mechanical limbs through a trocar located in the incision, and wherein a shortest distance between a base of the trocar and the surgical zone, measured along a wall of the body, is less than 10 cm, less than 8 cm, less than 5 cm, less than 4 cm, less than 3 cm, less than 2 cm, or less than 1 cm.

Embodiment 77. The method of any one of embodiments 55 to 76, wherein the inserting comprises, in order:
placing a trocar in the incision, while the trocar is in a first angular orientation relative to abdominal tissue including the incision;
inserting the mechanical limbs through the trocar to a first extent while the trocar is in the first angular orientation, such that a first segment of the articulated mechanical limb is disposed within the body;
changing an angular orientation of the trocar, together with the mechanical limbs, relative to the abdominal tissue; and
inserting the mechanical limbs through the trocar to a second extent while the trocar is in the second angular orientation, such that the first segment and an additional second segment of the mechanical limbs are disposed within the body.

Embodiment 78. The method of embodiment 77, wherein the first angular orientation is substantially perpendicular to the abdominal tissue, and the second angular orientation is at an acute angle relative to the abdominal tissue.

Embodiment 79. The method of any one of embodiments 55 to 78, wherein the inserting comprises inserting a plurality of the articulated mechanical limbs into the body through the incision, and the conducting of the abdominal surgery comprises conducting the abdominal surgery using the tools of the plurality of articulated mechanical limbs.

Embodiment 80. The method of any one of embodiments 55 to 79, wherein the inserting comprises inserting the at least one flexible portion into the body, while maintaining at least a majority of the linear portion outside of the body.

Embodiment 81. The method of any one of embodiments 55 to 79, wherein the inserting comprises inserting the at least one flexible portion into the body, while maintaining the entirety of the linear portion outside of the body.

Embodiment 82. The method of any one of embodiments 55 to 79, wherein a ratio between a length of the articulated mechanical limb disposed outside the body and a length of the articulated mechanical limb disposed within the body, measured as a sum of lengths of longitudinal axes of portions of the articulated mechanical limb within the body, is in the range of 1:2-1:4.

Embodiment 83. The method of any one of embodiments 55 to 79, wherein a ratio between a length of portions of the articulated mechanical limb disposed outside the body and a length of portions of the articulated mechanical limb disposed within the body, measured as a sum of lengths of longitudinal axes of portions of the articulated mechanical limb within the body, is at least 1:2 or at least 1:3.

Embodiment 84. The method of any one of embodiments 55 to 79, wherein a ratio between a length of the articulated mechanical limb, disposed within the body, which is stationary relative to the abdominal incision, and a length of the articulated mechanical limb disposed within the body which is movable relative to the abdominal incision, when the mount outside the body is stationary, is at least 1:5, at least 1:8, at least 1:10, or at least 1:15.

Embodiment 85. A method of carrying out abdominal surgery in a surgical zone of a body, the method comprising:
inserting an articulated mechanical limb into the body through an abdominal incision, the abdominal incision being outside of the surgical zone, where the articulated mechanical limb comprises:
   a linear portion, at least part of which extends outside the body, the linear portion being coupled to a mount disposed outside the body;
at least one flexible portion coupled to the linear portion; and
   a tool coupled to the at least one flexible portion;
bending the articulated mechanical limb within the body at the at least one flexible portion such that the tool thereof enters the surgical zone; and
using the tool, conducting surgery within the surgical zone,
wherein a shortest distance between the surgical zone and the abdominal incision, measured along a body wall, is less than 10 cm, less than 8 cm, less than 5 cm, less than 4 cm, less than 3 cm, less than 2 cm, or less than 1 cm.

Embodiment 86. The method of embodiment 85, wherein, during the bending, a sum of angles between adjacent effective segment longitudinal axes of the mechanical limb, in at least one three dimensionally oriented plane, is greater than 90°.

Embodiment 87. The method of any one of embodiments 85 to 86, wherein the abdominal surgery comprises hernia surgery, and wherein the conducting of the surgery comprises repairing a hernia in the surgical zone using the tool of the articulated mechanical limb.

Embodiment 88. The method of embodiment 87, wherein the repairing comprises covering the site of the hernia with a mesh.

Embodiment 89. The method of embodiment 88, further comprising, inserting the mesh into the body through the incision.

Embodiment 90. The method of any one of embodiments 87 to 89, wherein the repairing of the hernia comprises moving displaced tissue of the hernia body through a second tissue.

Embodiment 91. The method of any one of embodiments 87 to 90, wherein the hernia is an inguinal hernia, and the surgical zone is the inguinal triangle.

Embodiment 92. The method of embodiment 91, wherein the bending includes bending around the pubic bone of the body.

Embodiment 93. The method of any one of embodiments 87 to 90, wherein the hernia is a ventral hernia, and the surgical zone is an abdominal wall of the abdomen.

Embodiment 94. The method of embodiment any one of embodiments 85 to 93, wherein the bending comprises bending of the at least one flexible portion such that the tool is disposed above the horizon relative to the linear portion entering the abdominal incision.

Embodiment 95. The method of embodiment any one of embodiments 85 to 94, wherein the bending comprises bending of the at least one flexible portion such that the tool is disposed in a tool plane, the tool plane being above an incision plane formed by the abdominal incision and the linear portion, relative to a reference surface.

Embodiment 96. The method of any one of embodiments 85 to 95, wherein the bending of the articulated mechanical limb is such that a length of the articulated mechanical limb within the body, measured as a sum of lengths of longitudinal axes of portions of the articulated mechanical limb within the body, is at least 1.5 times, at least 2 times, at least 3 times, or at least 4 times the distance between the incision and the surgical zone, where the distance is a shortest distance between the incision and the surgical zone when measured along a body wall.

Embodiment 97. The method of any one of embodiments 85 to 96, wherein a length of the incision is in the range of 1 cm to 5 cm, 2 cm to 5 cm, 1 cm to 4 cm, 2 cm to 4 cm, 1 cm to 3 cm, or 2 cm to 3 cm.

Embodiment 98. The method of any one of embodiments 85 to 97, wherein the abdominal incision is a Pfannenstiel incision or a sub-Pfannenstiel incision.

Embodiment 99. The method of any one of embodiments 85 to 97, wherein the abdominal incision is an umbilical incision.

Embodiment 100. The method of any one of embodiments 85 to 99, wherein the bending of the articulated mechanical limb is to contact the surgical zone from an access direction where the access direction is different by at least 90° from a direction of entry of the articulated mechanical limb into the body.

Embodiment 101. The method of any one of embodiments 85 to 100, wherein the at least one flexible portion of the articulated mechanical limb comprises a first flexible portion coupled to the linear portion and a second flexible portion coupled at one end thereof the first flexible portion and at an opposing end thereof to the tool.

Embodiment 102. The method of embodiment 101, wherein each of the first and second flexible portions is independently rotatable about its longitudinal axis, and is independently bendable in a single bending plane.

Embodiment 103. The method of embodiment 101 or embodiment 102, wherein each of the first and second flexible portions is bendable in one rotational direction around each corresponding bending plane.

Embodiment 104. The method of any one of embodiments 85 to 103, wherein the conducting surgery comprises holding a portion of body tissue away from the surgical zone.

Embodiment 105. The method of any one of embodiments 85 to 104, further comprising inflating an abdominal cavity prior to, or during, the conducting of the surgery.

Embodiment 106. The method of any one of embodiments 85 to 105, wherein the inserting comprises, in order:
placing a trocar in the incision, while the trocar is in a first angular orientation relative to abdominal tissue including the incision;
inserting the mechanical limbs through the trocar to a first extent while the trocar is in the first angular orientation, such that a first segment of the articulated mechanical limb is disposed within the body;
changing an angular orientation of the trocar, together with the mechanical limbs, relative to the abdominal tissue; and
inserting the mechanical limbs through the trocar to a second extent while the trocar is in the second angular orientation, such that the first segment and an additional second segment of the mechanical limbs are disposed within the body.

Embodiment 107. The method of embodiment 106, wherein the first angular orientation is substantially perpendicular to the abdominal tissue, and the second angular orientation is at an acute angle relative to the abdominal tissue.

Embodiment 108. The method of any one of embodiments 85 to 107, wherein the inserting comprises inserting a plurality of the articulated mechanical limbs into the body through the incision, and the conducting of the abdominal surgery comprises conducting the abdominal surgery using the tools of the plurality of articulated mechanical limbs.

Embodiment 109. The method of any one of embodiments 85 to 108, wherein the inserting comprises inserting the at least one flexible portion into the body, while maintaining at least a majority of the linear portion outside of the body.

Embodiment 110. The method of any one of embodiments 85 to 108, wherein the inserting comprises inserting the at least one flexible portion into the body, while maintaining the entirety of the linear portion outside of the body.

Embodiment 111. The method of any one of embodiments 85 to 108, wherein a ratio between a length of the articulated mechanical limb disposed outside the body and a length of the articulated mechanical limb disposed within the body, measured as a sum of lengths of longitudinal axes of portions of the articulated mechanical limb within the body, is in the range of 1:2-1:4.

Embodiment 112. The method of any one of embodiments 85 to 108, wherein a ratio between a length of portions of the articulated mechanical limb disposed outside the body and a length of portions of the articulated mechanical limb disposed within the body, measured as a sum of lengths of longitudinal axes of portions of the articulated mechanical limb within the body, is at least 1:2 or at least 1:3.

Embodiment 113. The method of any one of embodiments 85 to 108, wherein a ratio between a length of the articulated mechanical limb, disposed within the body, which is stationary relative to the abdominal incision, and a length of the articulated mechanical limb disposed within the body which is movable relative to the abdominal incision, when the mount outside the body is stationary, is at least 1:5, at least 1:8, at least 1:10, or at least 1:15.

Embodiment 114. A method of carrying out abdominal surgery in a surgical zone of a body, the method comprising:
inserting an articulated mechanical limb into the body through an abdominal incision, the abdominal incision being outside of the surgical zone, where the articulated mechanical limb comprises:
   a linear portion, at least part of which extends outside the body, the linear portion being coupled to a mount disposed outside the body;
   at least one flexible portion coupled to the linear portion; and
   a tool coupled to the at least one flexible portion;
bending the articulated mechanical limb within the body at the at least one flexible portion such that the tool thereof enters the surgical zone; and
using the tool, conducting surgery within the surgical zone,
wherein a ratio between a length of the articulated mechanical limb within the body, measured as a sum of lengths of longitudinal axes of portions of the articulated mechanical limb within the body, and a distance between the abdominal incision and the surgical zone, is at least 2:1, the distance being a shortest distance between the abdominal incision and the surgical zone when measured along a body wall.

Embodiment 115. The method of embodiment 114, wherein the abdominal incision is a Pfannenstiel incision or a sub-Pfannenstiel incision.

Embodiment 116. The method of embodiment 114, wherein the abdominal incision is an umbilical incision.

Embodiment 117. The method of any one of embodiments 114 to 116, wherein, during the bending, a sum of angles between adjacent effective segment longitudinal axes of the articulated mechanical limb, in at least one three dimensionally oriented plane, is greater than 90°.

Embodiment 118. The method of any one of embodiments 114 to 117, wherein the abdominal surgery comprises hernia surgery, and wherein the conducting of the surgery comprises repairing a hernia in the surgical zone using the tool of the articulated mechanical limb.

Embodiment 119. The method of embodiment 118, wherein the repairing comprises covering the site of the hernia with a mesh.

Embodiment 120. The method of embodiment 119, further comprising, inserting the mesh into the body through the incision.

Embodiment 121. The method of any one of embodiments 118 to 120, wherein the repairing of the hernia comprises moving displaced tissue of the hernia body through a second tissue.

Embodiment 122. The method of any one of embodiments 118 to 121, wherein the hernia is an inguinal hernia, and the surgical zone is the inguinal triangle.

Embodiment 123. The method of embodiment 122, wherein the bending includes bending around the pubic bone of the body.

Embodiment 124. The method of any one of embodiments 118 to 121, wherein the hernia is a ventral hernia, and the surgical zone is in an abdominal wall.

Embodiment 125. The method of embodiment any one of embodiments 114 to 124, wherein the bending comprises bending of the at least one flexible portion such that the tool is disposed above the horizon relative to the linear portion entering the abdominal incision.

Embodiment 126. The method of embodiment any one of embodiments 114 to 125, wherein the bending comprises bending of the at least one flexible portion such that the tool is disposed in a tool plane, the tool plane being above an incision plane formed by the abdominal incision and the linear portion, relative to a reference surface.

Embodiment 127. The method of any one of embodiments 55 to 66, wherein the ratio between the length of the articulated mechanical arm within the body and the distance between the abdominal incision and the surgical zone is at least 3:1 or at least 4:1.

Embodiment 128. The method of any one of embodiments 114 to 127, wherein a length of the incision is in the range of 1 cm to 5 cm, 2 cm to 5 cm, 1 cm to 4 cm, 2 cm to 4 cm, 1 cm to 3 cm, or 2 cm to 3 cm.

Embodiment 129. The method of any one of embodiments 114 to 128, wherein the bending of the articulated mechanical limb is to contact the surgical zone from an access direction where the access direction is different by at least 90° from a direction of entry of the articulated mechanical limb into the body.

Embodiment 130. The method of any one of embodiments 114 to 129, wherein the at least one flexible portion of the articulated mechanical limb comprises a first flexible portion coupled to the linear portion and a second flexible portion coupled at one end thereof the first flexible portion and at an opposing end thereof to the tool.

Embodiment 131. The method of embodiment 130, wherein each of the first and second flexible portions is independently rotatable about its longitudinal axis, and is independently bendable in a single bending plane.

Embodiment 132. The method of embodiment 130 or embodiment 131, wherein each of the first and second flexible portions is bendable in one rotational direction around each corresponding bending plane.

Embodiment 133. The method of any one of embodiments 114 to 132, wherein the conducting surgery comprises holding a portion of body tissue away from the surgical zone.

Embodiment 134. The method of any one of embodiments 114 to 133, further comprising inflating an abdominal cavity prior to, or during, the conducting of the surgery.

Embodiment 135. The method of any one of embodiments 114 to 134, wherein the distance between the abdominal incision and the surgical zone is less than 10 cm, less than 8 cm, less than 5 cm, less than 4 cm, less than 3 cm, less than 2 cm, or less than 1 cm.

Embodiment 136. The method of any one of embodiments 114 to 135, wherein the inserting comprises, in order:
placing a trocar in the incision, while the trocar is in a first angular orientation relative to abdominal tissue including the incision;
inserting the mechanical limbs through the trocar to a first extent while the trocar is in the first angular orientation, such that a first segment of the articulated mechanical limb is disposed within the body;
changing an angular orientation of the trocar, together with the mechanical limbs, relative to the abdominal tissue; and
inserting the mechanical limbs through the trocar to a second extent while the trocar is in the second angular orientation, such that the first segment and an additional second segment of the mechanical limbs are disposed within the body.

Embodiment 137. The method of embodiment 136, wherein the first angular orientation is substantially perpendicular to the abdominal tissue, and the second angular orientation is at an acute angle relative to the abdominal tissue.

Embodiment 138. The method of any one of embodiments 114 to 137, wherein the inserting comprises inserting a plurality of the articulated mechanical limbs into the body through the incision, and the conducting of the abdominal surgery comprises conducting the abdominal surgery using the tools of the plurality of articulated mechanical limbs.

Embodiment 139. The method of any one of embodiments 114 to 137, wherein the inserting comprises inserting the at least one flexible portion into the body, while maintaining at least a majority of the linear portion outside of the body.

Embodiment 140. The method of any one of embodiments 114 to 137, wherein the inserting comprises inserting the at least one flexible portion into the body, while maintaining the entirety of the linear portion outside of the body.

Embodiment 141. The method of any one of embodiments 114 to 137, wherein a ratio between a length of the articulated mechanical limb disposed outside the body and a length of the articulated mechanical limb disposed within the body, measured as a sum of lengths of longitudinal axes of portions of the articulated mechanical limb within the body, is in the range of 1:2-1:4.

Embodiment 142. The method of any one of embodiments 114 to 137, wherein a ratio between a length of portions of the articulated mechanical limb disposed outside the body and a length of portions of the articulated mechanical limb disposed within the body, measured as a sum of lengths of longitudinal axes of portions of the articulated mechanical limb within the body, is at least 1:2 or at least 1:3.

Embodiment 143. The method of any one of embodiments 114 to 137, wherein a ratio between a length of the articulated mechanical limb, disposed within the body, which is stationary relative to the abdominal incision, and a length of the articulated mechanical limb disposed within the body which is movable relative to the abdominal incision, when the mount outside the body is stationary, is at least 1:5, at least 1:8, at least 1:10, or at least 1:15.

Embodiment 144. A method of carrying out abdominal surgery in a surgical zone of a body, the method comprising:
inserting an articulated mechanical limb into the body through an abdominal incision, where the articulated mechanical limb comprises:
   a linear portion, at least part of which extends outside the body, the linear portion being coupled to a mount disposed outside the body;
at least one flexible portion coupled to the linear portion; and
   a tool coupled to the at least one flexible portion;
bending the articulated mechanical limb within the body at the at least one flexible portion such that the tool thereof enters the surgical zone, wherein, following the bending, the tool is disposed above the horizon relative to the linear portion entering the abdominal incision; and
using the tool, conducting surgery within the surgical zone.

Embodiment 145. A method of carrying out abdominal surgery in a surgical zone of a body, the method comprising:
inserting an articulated mechanical limb into the body through an abdominal incision, where the articulated mechanical limb comprises:
   a linear portion, at least part of which extends outside the body, the linear portion being coupled to a mount disposed outside the body;
   at least one flexible portion coupled to the linear portion; and
   a tool coupled to the at least one flexible portion;
bending the articulated mechanical limb within the body at the at least one flexible portion such that the tool thereof enters the surgical zone, wherein, following the bending, the tool is disposed in a tool plane, the tool plane being above an incision plane formed by the abdominal incision and the linear portion, relative to a reference surface; and
using the tool, conducting surgery within the surgical zone.

Embodiment 146. The method of embodiment 144 or embodiment 145, wherein the abdominal incision is a Pfannenstiel incision or a sub-Pfannenstiel incision.

Embodiment 147. The method of embodiment 144 or embodiment 145, wherein the abdominal incision is an umbilical incision.

Embodiment 148. The method of embodiment any one of embodiments 144 to 147, wherein, during the bending, a sum of angles between adjacent effective segment longitudinal axes of the articulated mechanical limb, in at least one three dimensionally oriented plane, is greater than 90°.

Embodiment 149. The method of any one of embodiments 144 to 148, wherein the abdominal surgery comprises hernia surgery, and wherein the conducting of the surgery comprises repairing a hernia in the surgical zone using the tool of the articulated mechanical limb.

Embodiment 150. The method of embodiment 149, wherein the repairing comprises covering the site of the hernia with a mesh.

Embodiment 151. The method of embodiment 150, further comprising, inserting the mesh into the body through the incision.

Embodiment 152. The method of any one of embodiments 149 to 151, wherein the repairing of the hernia comprises moving displaced tissue of the hernia body through a second tissue.

Embodiment 153. The method of any one of embodiments 144 to 152, wherein the abdominal incision is outside of the abdominal surgical zone, the bending of the articulated mechanical limb is such that a length of the articulated mechanical limb within the body, measured as a sum of lengths of longitudinal axes of portions of the articulated mechanical limb within the body, is at least 1.5 times, at least 2 times, at least 3 times, or at least 4 times the distance between the incision and the surgical zone, where the distance is a shortest distance between the incision and the surgical zone when measured along a body wall.

Embodiment 154. The method of any one of embodiments 144 to 153, wherein a length of the incision is in the range of 1 cm to 5 cm, 2 cm to 5 cm, 1 cm to 4 cm, 2 cm to 4 cm, 1 cm to 3 cm, or 2 cm to 3 cm.

Embodiment 155. The method of any one of embodiments 144 to 154, wherein the bending of the articulated mechanical limb is to contact the surgical zone from an access direction where the access direction is different by at least 90° from a direction of entry of the articulated mechanical limb into the body.

Embodiment 156. The method of any one of embodiments 144 to 155, wherein the at least one flexible portion of the articulated mechanical limb comprises a first flexible portion coupled to the linear portion and a second flexible portion coupled at one end thereof the first flexible portion and at an opposing end thereof to the tool.

Embodiment 157. The method of embodiment 156, wherein each of the first and second flexible portions is independently rotatable about its longitudinal axis, and is independently bendable in a single bending plane.

Embodiment 158. The method of embodiment 156 or embodiment 157, wherein each of the first and second flexible portions is bendable in one rotational direction around each corresponding bending plane.

Embodiment 159. The method of any one of embodiments 144 to 158, wherein the conducting surgery comprises holding a portion of body tissue away from the surgical zone.

Embodiment 160. The method of any one of embodiments 144 to 159, further comprising inflating an abdominal cavity prior to, or during, the conducting of the surgery.

Embodiment 161. The method of any one of embodiments 144 to 160, wherein the abdominal incision is outside of the surgical zone, and a shortest distance between the incision and the surgical zone, measured along a wall of the body, is less than 10 cm, less than 8 cm, less than 5 cm, less than 4 cm, less than 3 cm, less than 2 cm, or less than 1 cm.

Embodiment 162. The method of any one of embodiments 144 to 160, wherein the abdominal incision is outside of the surgical zone, the inserting comprises inserting the mechanical limbs through a trocar located in the incision, and wherein a shortest distance between a base of the trocar and the surgical zone, measured along a wall of the body, is less than 10 cm, less than 8 cm, less than 5 cm, less than 4 cm, less than 3 cm, less than 2 cm, or less than 1 cm.

Embodiment 163. The method of any one of embodiments 144 to 162, wherein the inserting comprises, in order:
placing a trocar in the incision, while the trocar is in a first angular orientation relative to abdominal tissue including the incision;
inserting the mechanical limbs through the trocar to a first extent while the trocar is in the first angular orientation, such that a first segment of the articulated mechanical limb is disposed within the body;
changing an angular orientation of the trocar, together with the mechanical limbs, relative to the abdominal tissue; and
inserting the mechanical limbs through the trocar to a second extent while the trocar is in the second angular orientation, such that the first segment and an additional second segment of the mechanical limbs are disposed within the body.

Embodiment 164. The method of embodiment 163, wherein the first angular orientation is substantially perpendicular to the abdominal tissue, and the second angular orientation is at an acute angle relative to the abdominal tissue.

Embodiment 165. The method of any one of embodiments 144 to 164, wherein the inserting comprises inserting a plurality of the articulated mechanical limbs into the body through the incision, and the conducting of the abdominal surgery comprises conducting the abdominal surgery using the tools of the plurality of articulated mechanical limbs.

Embodiment 166. The method of any one of embodiments 144 to 165, wherein the inserting comprises inserting the at least one flexible portion into the body, while maintaining at least a majority of the linear portion outside of the body.

Embodiment 167. The method of any one of embodiments 144 to 165, wherein the inserting comprises inserting the at least one flexible portion into the body, while maintaining the entirety of the linear portion outside of the body.

Embodiment 168. The method of any one of embodiments 144 to 165, wherein a ratio between a length of the articulated mechanical limb disposed outside the body and a length of the articulated mechanical limb disposed within the body, measured as a sum of lengths of longitudinal axes of portions of the articulated mechanical limb within the body, is in the range of 1:2-1:4.

Embodiment 169. The method of any one of embodiments 144 to 165, wherein a ratio between a length of portions of the articulated mechanical limb disposed outside the body and a length of portions of the articulated mechanical limb disposed within the body, measured as a sum of lengths of longitudinal axes of portions of the articulated mechanical limb within the body, is at least 1:2 or at least 1:3.

Embodiment 170. The method of any one of embodiments 144 to 165, wherein a ratio between a length of the articulated mechanical limb, disposed within the body, which is stationary relative to the abdominal incision, and a length of the articulated mechanical limb disposed within the body which is movable relative to the abdominal incision, when the mount outside the body is stationary, is at least 1:5, at least 1:8, at least 1:10, or at least 1:15.

Embodiment 171. A method of carrying out abdominal surgery in a surgical zone of a body, the method comprising:
inserting an articulated mechanical limb into the body through an abdominal incision, where the articulated mechanical limb comprises:
   a linear portion, at least part of which extends outside the body, the linear portion being coupled to a mount disposed outside the body;
   at least one flexible portion coupled to the linear portion; and
   a tool coupled to the at least one flexible portion;
bending the articulated mechanical limb within the body at the at least one flexible portion such that the tool thereof enters the surgical zone; and
using the tool, conducting surgery within the surgical zone,
wherein the inserting comprises inserting the first and second flexible portions into the body, while maintaining at least a majority of the linear portion outside of the body.

Embodiment 172. The method of embodiment 171, wherein the inserting comprises inserting the first and second flexible portions into the body, while maintaining the entirety of the linear portion outside of the body.

Embodiment 173. A method of carrying out abdominal surgery in a surgical zone of a body, the method comprising:
inserting an articulated mechanical limb into the body through an abdominal incision, where the articulated mechanical limb comprises:
   a linear portion, at least part of which extends outside the body, the linear portion being coupled to a mount disposed outside the body;
   at least one flexible portion coupled to the linear portion; and
   a tool coupled to the at least one flexible portion;
bending the articulated mechanical limb within the body at the at least one flexible portion such that the tool thereof enters the surgical zone; and
using the tool, conducting surgery within the surgical zone,
wherein a ratio between a length of the articulated mechanical limb disposed outside the body and a length of the articulated mechanical limb disposed within the body, measured as a sum of lengths of longitudinal axes of portions of the articulated mechanical limb within the body, is in the range of 1:2-1:4.

Embodiment 174. A method of carrying out abdominal surgery in a surgical zone of a body, the method comprising:
inserting an articulated mechanical limb into the body through an abdominal incision, where the articulated mechanical limb comprises:
   a linear portion, at least part of which extends outside the body, the linear portion being coupled to a mount disposed outside the body;
   at least one flexible portion coupled to the linear portion; and
   a tool coupled to the at least one flexible portion;
bending the articulated mechanical limb within the body at the at least one flexible portion such that the tool thereof enters the surgical zone; and
using the tool, conducting surgery within the surgical zone, wherein a ratio between a length of portions of the articulated mechanical limb disposed outside the body and a length of portions of the articulated mechanical limb disposed within the body, measured as a sum of lengths of longitudinal axes of portions of the articulated mechanical limb within the body, is at least 1:2 or at least 1:3.

Embodiment 175. A method of carrying out abdominal surgery in a surgical zone of a body, the method comprising:
inserting an articulated mechanical limb into the body through an abdominal incision, where the articulated mechanical limb comprises:
   a linear portion, at least part of which extends outside the body, the linear portion being coupled to a mount disposed outside the body;
   at least one flexible portion coupled to the linear portion; and
   a tool coupled to the at least one flexible portion;
bending the articulated mechanical limb within the body at the at least one flexible portion such that the tool thereof enters the surgical zone; and
using the tool, conducting surgery within the surgical zone, wherein a ratio between a length of the articulated mechanical limb, disposed within the body, which is stationary relative to the abdominal incision, and a length of the articulated mechanical limb disposed within the body which is movable relative to the abdominal incision, when the mount outside the body is stationary, is at least 1:5, at least 1:8, at least 1:10, or at least 1:15.

Embodiment 176. The method of any one of embodiments 171 to 175, wherein the abdominal incision is a Pfannenstiel incision or a sub-Pfannenstiel incision.

Embodiment 177. The method of any one of embodiments 171 to 175, wherein the abdominal incision is an umbilical incision.

Embodiment 178. The method of any one of embodiments 171 to 177, wherein, during the bending, a sum of angles between adjacent effective segment longitudinal axes of the articulated mechanical limb, in at least one three dimensionally oriented plane, is greater than 90°.

Embodiment 179. The method of any one of embodiments 171 to 178, wherein the abdominal surgery comprises hernia surgery, and wherein the conducting of the surgery comprises repairing a hernia in the surgical zone using the tool of the articulated mechanical limb.

Embodiment 180. The method of embodiment 179, wherein the repairing comprises covering the site of the hernia with a mesh.

Embodiment 181. The method of embodiment 180, further comprising, inserting the mesh into the body through the incision.

Embodiment 182. The method of any one of embodiments 179 to 181, wherein the repairing of the hernia comprises moving displaced tissue of the hernia body through a second tissue.

Embodiment 183. The method of any one of embodiments 179 to 182, wherein the hernia is an inguinal hernia, and the surgical zone is the inguinal triangle.

Embodiment 184. The method of embodiment 183, wherein the bending includes bending around the pubic bone of the body.

Embodiment 185. The method of any one of embodiments 179 to 182, wherein the hernia is a ventral hernia, and the surgical zone is an abdominal wall.

Embodiment 186. The method of embodiment any one of embodiments 171 to 185, wherein the bending comprises bending of the at least one flexible portion such that the tool is disposed above the horizon relative to the linear portion entering the abdominal incision.

Embodiment 187. The method of embodiment any one of embodiments 171 to 186, wherein the bending comprises bending of the at least one flexible portion such that the tool is disposed in a tool plane, the tool plane being above an incision plane formed by the abdominal incision and the linear portion, relative to a reference surface.

Embodiment 188. The method of any one of embodiments 171 to 187, wherein the abdominal incision is outside of the abdominal surgical zone, the bending of the articulated mechanical limb is such that a length of the articulated mechanical limb within the body, measured as a sum of lengths of longitudinal axes of portions of the articulated mechanical limb within the body, is at least 1.5 times, at least 2 times, at least 3 times, or at least 4 times the distance between the incision and the surgical zone, where the distance is a shortest distance between the incision and the surgical zone when measured along a body wall.

Embodiment 189. The method of any one of embodiments 171 to 188, wherein a length of the incision is in the range of 1 cm to 5 cm, 2 cm to 5 cm, 1 cm to 4 cm, 2 cm to 4 cm, 1 cm to 3 cm, or 2 cm to 3 cm.

Embodiment 190. The method of any one of embodiments 171 to 189, wherein the bending of the articulated mechanical limb is to contact the surgical zone from an access direction where the access direction is different by at least 90° from a direction of entry of the articulated mechanical limb into the body.

Embodiment 191. The method of any one of embodiments 171 to 190, wherein the at least one flexible portion of the articulated mechanical limb comprises a first flexible portion coupled to the linear portion and a second flexible portion coupled at one end thereof the first flexible portion and at an opposing end thereof to the tool.

Embodiment 192. The method of embodiment 191, wherein each of the first and second flexible portions is independently rotatable about its longitudinal axis, and is independently bendable in a single bending plane.

Embodiment 193. The method of embodiment 191 or embodiment 192, wherein each of the first and second flexible portions is bendable in one rotational direction around each corresponding bending plane.

Embodiment 194. The method of any one of embodiments 171 to 193, wherein the conducting surgery comprises holding a portion of body tissue away from the surgical zone.

Embodiment 195. The method of any one of embodiments 171 to 194, further comprising inflating an abdominal cavity prior to, or during, the conducting of the surgery.

Embodiment 196. The method of any one of embodiments 171 to 195, wherein the abdominal incision is outside of the surgical zone, and a shortest distance between the incision and the surgical zone, measured along a wall of the body, is less than 10 cm, less than 8 cm, less than 5 cm, less than 4 cm, less than 3 cm, less than 2 cm, or less than 1 cm.

Embodiment 197. The method of any one of embodiments 171 to 195, wherein the abdominal incision is outside of the surgical zone, the inserting comprises inserting the mechanical limbs through a trocar located in the incision, and wherein a shortest distance between a base of the trocar and the surgical zone, measured along a wall of the body, is less than 10 cm, less than 8 cm, less than 5 cm, less than 4 cm, less than 3 cm, less than 2 cm, or less than 1 cm.

Embodiment 198. The method of any one of embodiments 171 to 197, wherein the inserting comprises, in order:
placing a trocar in the incision, while the trocar is in a first angular orientation relative to abdominal tissue including the incision;
inserting the mechanical limbs through the trocar to a first extent while the trocar is in the first angular orientation, such that a first segment of the articulated mechanical limb is disposed within the body;
changing an angular orientation of the trocar, together with the mechanical limbs, relative to the abdominal tissue; and
inserting the mechanical limbs through the trocar to a second extent while the trocar is in the second angular orientation, such that the first segment and an additional second segment of the mechanical limbs are disposed within the body.

Embodiment 199. The method of embodiment 198, wherein the first angular orientation is substantially perpendicular to the abdominal tissue, and the second angular orientation is at an acute angle relative to the abdominal tissue.

Embodiment 200. The method of any one of embodiments 171 to 199, wherein the inserting comprises inserting a plurality of the articulated mechanical limbs into the body through the incision, and the conducting of the abdominal surgery comprises conducting the abdominal surgery using the tools of the plurality of articulated mechanical limbs.

Embodiment 201. A method of carrying out abdominal surgery using a mechanical limb including (i) a linear portion, (ii) at least one flexible portion coupled to the linear portion, and (iii) a tool coupled to the second flexible portion, the method comprising:
placing a trocar in the abdominal incision, while the trocar is in a first angular orientation relative to abdominal tissue including the incision;
inserting a mechanical limb into a body through the trocar to a first extent while the trocar is in the first angular orientation, such that a first segment of the mechanical limb is disposed within the body;
changing an angular orientation of the trocar, together with the first segment of the mechanical limb, relative to the abdominal tissue;
inserting the mechanical limb through the trocar to a second extent while the trocar is in the second angular orientation, such that the first segment and an additional second segment of the mechanical limb are disposed within the body;
bending the mechanical limb within the body at the first and the second flexible portions such that the tool thereof enters an abdominal surgical zone; and
using the tool, conducting surgery within the abdominal surgical zone.

Embodiment 202. A method of conducting abdominal surgery in a surgical zone, the method comprising:
creating an incision at a sub-Pfannenstiel region of the abdomen;
inserting a mechanical limb into a body of a subject through the incision, where the mechanical limb comprises a linear portion, at least part of which extends outside the body, and a tool coupled to a distal end of the mechanical limb;
manipulating the mechanical limb within the body so that the tool contacts the abdominal target site; and
using the tool, conducting the abdominal surgery within the surgical zone.

Embodiment 203. The method of embodiment 202, wherein the creating of the incision includes creating the incision in close proximity (e.g. up to one finger's breadth) to the pubic bone, below the Pfannenstiel line.

Embodiment 204. The method of embodiment 202, wherein the mechanical limb includes a straight mechanical limb, such that the tool is coupled to a distal end of the linear portion.

Embodiment 205. The method of embodiment 202, wherein the mechanical limb includes an articulated mechanical limb, further including at least one flexible portion disposed between the linear portion and the tool.

Embodiment 206. The method of embodiment 205, wherein the manipulating includes bending the articulated mechanical limb within the body at the at least one flexible region so that the tool contacts the surgical zone.

Embodiment 207. The method of embodiment 206, wherein the bending includes bending the articulated mechanical limb such that a sum of angles between adjacent effective segment longitudinal axes, in at least one three dimensionally oriented plane, is greater than 90°.

Embodiment 208. The method of embodiment 206 or embodiment 207, wherein the bending includes bending around the pubic bone of the body.

Embodiment 209. The method of embodiment 206 or embodiment 207, wherein the bending includes bending of the at least one flexible portion such that the tool is disposed above the horizon relative to the linear portion entering the incision.

Embodiment 210. The method of embodiment 209, wherein the bending includes bending of the at least one flexible portion such that the tool is disposed in a tool plane, the tool plane being above an incision plane formed by the incision and the linear portion, relative to a reference surface.

Embodiment 211. The method of any one of embodiments 206 to 210, wherein the bending of the articulated mechanical limb is to contact the surgical zone from an access direction where the access direction is different by at least 90° from a direction of entry of the articulated mechanical limb into the body.

Embodiment 212. The method of any one of embodiments 205 to 211, wherein the at least one flexible portion of the articulated mechanical limb includes a first flexible portion coupled to the linear portion and a second flexible portion coupled at one end thereof the first flexible portion and at an opposing end thereof to the tool.

Embodiment 213. The method of embodiment 212, wherein each of the first and second flexible portions is independently rotatable about its longitudinal axis, and is independently bendable in a single bending plane.

Embodiment 214. The method of any one of embodiments 212 to 213, wherein each of the first and second flexible portions is bendable in one rotational direction around each corresponding bending plane.

Embodiment 215. The method of any one of embodiments 205 to 214, wherein the inserting includes inserting the at least one flexible portion into the body, while maintaining at least a majority of the linear portion outside of the body.

Embodiment 216. The method of any one of embodiments 205 to 214, wherein the inserting includes inserting the at least one flexible portion into the body, while maintaining the entirety of the linear portion outside of the body.

Embodiment 217. The method of any one of embodiments 202 to 216, wherein the manipulating includes repairing a hernia with the tool of the mechanical limb.

Embodiment 218. The method of embodiment 217, wherein the repairing of the hernia includes moving displaced tissue of the hernia body through a second tissue.

Embodiment 219. The method of embodiment 217 or embodiment 218, wherein the repairing of the hernia includes covering a site of the hernia with a mesh.

Embodiment 220. The method of embodiment 219, further including inserting the mesh into the body through the incision.

Embodiment 221. The method of any one of embodiments 217 to 220, wherein the hernia is an inguinal hernia, and the surgical zone is the inguinal triangle.

Embodiment 222. The method of any one of embodiments 217 to 220, wherein the hernia is a ventral hernia, and the surgical zone is a surface of the abdominal wall.

Embodiment 223. The method of any one of embodiments 202 to 216, wherein the manipulating includes conducting a hysterectomy using the tool of the mechanical limb.

Embodiment 224. The method of any one of embodiments 202 to 216, wherein the manipulating includes conducting a colectomy using the tool of the mechanical limb.

Embodiment 225. The method of any one of embodiments 202 to 216, wherein the manipulating includes conducting a sleeve gastrectomy using the tool of the mechanical limb.

Embodiment 226. The method of any one of embodiments 202 to 216, wherein the manipulating includes conducting a cholecystectomy using the tool of the mechanical limb.

Embodiment 227. The method of any one of embodiments 202 to 216, wherein the manipulating includes conducting an appendectomy using the tool of the mechanical limb.

Embodiment 228. The method of any one of embodiments 202 to 216, wherein the manipulating includes conducting a Nissen fundoplication using the tool of the mechanical limb.

Embodiment 229. The method of any one of embodiments 202 to 216, wherein the manipulating includes conducting a gastric bypass surgery using the tool of the mechanical limb.

Embodiment 230. The method of any one of embodiments 202 to 229, wherein a length of the incision is in the range of 1 cm to 5 cm, 2 cm to 5 cm, 1 cm to 4 cm, 2 cm to 4 cm, 1 cm to 3 cm, or 2 cm to 3 cm.

Embodiment 231. The method of any one of embodiments 202 to 230, wherein the repairing includes holding a portion of body tissue away from the surgical zone.

Embodiment 232. The method of any one of embodiments 202 to 231, wherein the inserting includes inserting a plurality of the mechanical limbs into the body through the incision, and the manipulating includes manipulating the tools of the plurality of mechanical limbs to conduct the surgery.

Embodiment 233. The method of any one of embodiments 202 to 232, further including inflating an abdominal cavity prior to, or during, the manipulating.

Embodiment 234. The method of any one of embodiments 202 to 233, wherein the incision is outside of the surgical zone, and a shortest distance between the incision and the surgical zone, measured along a wall of the body, is less than 10 cm.

Embodiment 235. The method of any one of embodiments 202 to 234, wherein a ratio between a length of the mechanical limb disposed outside the body and a length of the mechanical limb disposed within the body, measured as a sum of lengths of longitudinal axes of portions of the mechanical limb within the body, is in the range of 1:2-1:4.

Embodiment 236. The method of any one of embodiments 202 to 234, wherein a ratio between a length of portions of the mechanical limb disposed outside the body and a length of portions of the mechanical limb disposed within the body, measured as a sum of lengths of longitudinal axes of portions of the mechanical limb within the body, is at least 1:2 or at least 1:3.

Embodiment 237. The method of any one of embodiments 202 to 236, wherein the inserting includes, in order:
placing a trocar in the incision, while the trocar is in a first angular orientation relative to abdominal tissue including the incision;
inserting the mechanical limb through the trocar to a first extent while the trocar is in the first angular orientation, such that a first segment of the mechanical limb is disposed within the body;
changing an angular orientation of the trocar, together with the mechanical limb, relative to the abdominal tissue; and
inserting the mechanical limb through the trocar to a second extent while the trocar is in the second angular orientation, such that the first segment and an additional second segment of the mechanical limb are disposed within the body.

Embodiment 238. The method of embodiment 237, wherein the first angular orientation is substantially perpendicular to the abdominal tissue, and the second angular orientation is at an acute angle relative to the abdominal tissue.

Embodiment 239. The method of any one of embodiments 202 to 238, where the manipulating of the mechanical limb does not affect the shape or extension of a portion of the mechanical limb disposed outside the body of the subject

It will be appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A method of conducting abdominal surgery in a surgical zone, the method comprising:
creating an incision at a sub-Pfannenstiel region of the abdomen; inserting a mechanical limb into a body of a subject through the incision, where the mechanical limb comprises a linear portion, at least part of which extends outside the body, and a tool coupled to a distal end of the mechanical limb;
manipulating the mechanical limb within the body so that the tool contacts the abdominal target site; and
using the tool, conducting the abdominal surgery within the surgical zone.

2. The method of claim 1, wherein the creating of the incision comprises creating the incision in close proximity to the pubic bone, below the Pfannenstiel line.

3. (canceled)

4. The method of claim 1, wherein the mechanical limb comprises an articulated mechanical limb, further including at least one flexible portion disposed between the linear portion and the tool.

5. The method of claim 4, wherein the manipulating comprises bending the articulated mechanical limb within the body at the at least one flexible region so that the tool contacts the surgical zone.

6. The method of claim 5, wherein the bending comprises bending the articulated mechanical limb such that a sum of angles between adjacent effective segment longitudinal axes, in at least one three dimensionally oriented plane, is greater than 90°.

7. The method of claim 5, wherein the bending comprises bending around the pubic bone of the body.

8. (canceled)

9. (canceled)

10. The method of claim 5, wherein the bending of the articulated mechanical limb is to contact the surgical zone from an access direction where the access direction is different by at least 90° from a direction of entry of the articulated mechanical limb into the body.

11. (canceled)

12. (canceled)

13. (canceled)

14. The method of claim 4, wherein the inserting comprises inserting the at least one flexible portion into the body, while maintaining at least a majority of the linear portion outside of the body.

15. (canceled)

16. The method of claim 1, wherein the manipulating comprises repairing a hernia with the tool of the mechanical limb.

17. The method of claim 16, wherein the repairing of the hernia comprises moving displaced tissue of the hernia body through a second tissue.

18. (canceled)

19. (canceled)

20. The method of claim 16, wherein the hernia is an inguinal hernia, and the surgical zone is the inguinal triangle.

21. The method of claim 16, wherein the hernia is a ventral hernia, and the surgical zone is a surface of the abdominal wall.

22. (canceled)

23. (canceled)

24. (canceled)

25. (canceled)

26. (canceled)

27. (canceled)

28. (canceled)

29. The method of claim 1, wherein a length of the incision is in the range of 1 cm to 5 cm, 2 cm to 5 cm, 1 cm to 4 cm, 2 cm to 4 cm, 1 cm to 3 cm, or 2 cm to 3 cm.

30. The method of claim 1, wherein the repairing comprises holding a portion of body tissue away from the surgical zone.

31. (canceled)

32. (canceled)

33. The method of claim 1, wherein the incision is outside of the surgical zone, and a shortest distance between the incision and the surgical zone, measured along a wall of the body, is less than 10 cm.

34. The method of claim 1, wherein a ratio between a length of the mechanical limb disposed outside the body and a length of the mechanical limb disposed within the body, measured as a sum of lengths of longitudinal axes of portions of the mechanical limb within the body, is in the range of 1:2-1:4.

35. The method of claim 1, wherein a ratio between a length of portions of the mechanical limb disposed outside the body and a length of portions of the mechanical limb disposed within the body, measured as a sum of lengths of longitudinal axes of portions of the mechanical limb within the body, is at least 1:2 or at least 1:3.

36. The method of claim 1, wherein the inserting comprises, in order:
placing a trocar in the incision, while the trocar is in a first angular orientation relative to abdominal tissue including the incision;
inserting the mechanical limb through the trocar to a first extent while the trocar is in the first angular orientation, such that a first segment of the mechanical limb is disposed within the body;
changing an angular orientation of the trocar, together with the mechanical limb, relative to the abdominal tissue; and
inserting the mechanical limb through the trocar to a second extent while the trocar is in the second angular orientation, such that the first segment and an additional second segment of the mechanical limb are disposed within the body.

37. The method of claim 36, wherein the first angular orientation is substantially perpendicular to the abdominal tissue, and the second angular orientation is at an acute angle relative to the abdominal tissue.

38. (canceled)
